(19) United States
(12) Patent Application Publication
(10) Pub. No.: US 2018/0306616 A1
(43) Pub. Date: Oct. 25, 2018

(54) Fluid Flow Monitoring and Management Devices, Systems, and Methods

(71) Applicant: LogiLube, LLC, Fort Collins, CO (US)

(72) Inventors: William J. Gillette, II, Fort Collins, CO (US); Charles E. Ogden, Laramie, WY (US)

(73) Assignee: LogiLube, LLC, Fort Collins, CO (US)

(21) Appl. No.: 15/769,712

(22) PCT Filed: Oct. 20, 2016
(86) PCT No.: PCT/US16/57994

§ 371 Date: Apr. 19, 2018

Related U.S. Application Data
(60) Provisional application No. 62/244,148, filed on Oct. 20, 2015.

(51) Int. Cl. G01F 1/56; G01N 33/28

(52) U.S. Cl. CPC G01F 1/56; F04C 28/28; G01N 2291/02836; G01N 33/2888

(57) ABSTRACT

A fluid flow monitoring and management device that includes a fluid flow body having a magnet and a sensor secured to the fluid flow body. The sensor includes a magnetometer in a signal relationship with the magnet.

### CROSS REFERENCE TO RELATED APPLICATION

This application claims priority to U.S. Provisional Patent Application No. 62/244,146 which was filed on Oct. 20, 2015 titled “Fluid Flow Monitoring and Management Devices, Systems, and Methods”, the disclosure of which is incorporated herein by reference.

### TECHNICAL FIELD

The inventions pertain to fluid flow monitoring and management devices, systems, and methods.

### BACKGROUND OF THE DISCLOSURE

Fluid flow is the “life blood” of equipment and machinery. However, as the quality of the fluid flow goes; goes the equipment/machinery. That is, the health of the equipment/machinery depends on the fluid flow. If the fluid flow is compromised or diminished, the “health” of the equipment/machinery is compromised or diminished which ultimately can lead to a catastrophic failure of the equipment/machinery. Consequently, there always is a need for improved fluid flow monitoring and management devices, systems and methods to predict and prevent the diminishing of the health of the fluid flow, and correspondingly, to predict and prevent the failure of the equipment and machines in which the fluid flows.

### BRIEF DESCRIPTION OF THE DRAWINGS

Embodiments of the disclosure are described below with reference to the following accompanying drawings.

FIG. 1 is a flow diagram of an exemplary aspect of the invention;

FIG. 2 is a flow diagram of an exemplary aspect of the invention;

FIG. 3 is a side view of a compressor package;

FIG. 4 is a flow diagram of an exemplary aspect of the invention;

FIG. 5 is a flow diagram of an exemplary aspect of the invention;

FIG. 6 is a flow diagram of an exemplary aspect of the invention;

FIG. 7 is a flow diagram of an exemplary aspect of the invention;

FIG. 8 is an algorithm diagram of an exemplary aspect of the invention;

FIG. 9 is a schematic of oil consumption according to an exemplary aspect of the invention;

FIG. 10 is a schematic of oil consumption according to an exemplary aspect of the invention;

FIG. 11 is a schematic of oil consumption according to an exemplary aspect of the invention;

FIG. 12 is a schematic of oil consumption according to an exemplary aspect of the invention;

FIG. 13 is a schematic of oil consumption according to an exemplary aspect of the invention;

FIG. 14 is a schematic of oil consumption according to an exemplary aspect of the invention;

FIG. 15 is a schematic of oil consumption according to an exemplary aspect of the invention;

FIG. 16 is a schematic of oil consumption according to an exemplary aspect of the invention;

FIG. 17 is a schematic of oil consumption according to an exemplary aspect of the invention;

FIG. 18 is a schematic of oil consumption according to an exemplary aspect of the invention;

FIG. 19 is a schematic of oil consumption according to an exemplary aspect of the invention;

FIG. 20 is a schematic of oil consumption according to an exemplary aspect of the invention;

FIG. 21 is a partial view of an oil monitoring system according to an exemplary aspect of the invention;

FIGS. 22-23 are partial views of an oil level controller according to an exemplary aspect of the invention;

FIG. 24 is a partial view of an oil level controller installation;

FIG. 25 is a table of an oil level controller response;

FIG. 26 is a schematic of oil consumption according to an exemplary aspect of the invention;

FIGS. 27-28 are perspective views of a fluid flow monitoring and management device according to an exemplary aspect of the invention;

FIG. 29 is a perspective view of a sensor of the fluid flow monitoring and management device of FIGS. 27-28 according to an exemplary aspect of the invention;

FIG. 30 is an electronics layout of the sensor of FIG. 29 according to an exemplary aspect of the invention;

FIG. 31 is an exploded view of the body portion of the fluid flow monitoring and management device of FIGS. 27-28 according to an exemplary aspect of the invention;

FIG. 32 is a sectional view of the fluid flow monitoring and management device of FIGS. 27-28 according to an exemplary aspect of the invention;

FIG. 33 is a graphical representation of operation of the fluid flow monitoring and management device of FIGS. 27-28 according to an exemplary aspect of the invention;

FIG. 34 is a perspective view of the fluid flow monitoring and management device of FIGS. 27-28 mounted onto a compressor package according to an exemplary aspect of the invention;

FIGS. 35-36 are views of the fluid flow monitoring and management device of FIGS. 27-28 mounted onto a force-feed lube oil pump according to an exemplary aspect of the invention;

FIG. 37 is a sectional view of an exemplary level measure device according to an exemplary aspect of the invention;

FIG. 38 is a view of a differential pressure measurement in operation across a filter;

FIG. 39 is a perspective view of an exemplary fluid flow monitoring and management system according to an exemplary aspect of the invention;

FIG. 40 is a view of the exemplary fluid flow monitoring and management system of FIG. 39 mounted on a compressor package according to an exemplary aspect of the invention; and

FIG. 41 is an operation diagram of the exemplary fluid flow monitoring and management system of FIG. 39 according to an exemplary aspect of the invention.

### DETAILED DESCRIPTION OF THE EMBODIMENTS

This disclosure is submitted in furtherance of the constitutional purposes of the U.S. Patent Laws “to promote the progress of science and useful arts” (Article 1, Section 8).

The terms “a”, “an”, and “the” as used in the claims herein are used in conformance with long-standing claim drafting practice and not in a limiting way. Unless specifically set forth herein, the terms “a”, “an”, and “the” are not limited to one of such elements, but instead mean “at least one.”

### Anatomy of a Gas Compression Package

An engine driven gas compressor package is a device and/or system that is used very commonly in the oil and gas industry to “pump, or compress” natural gas so that it can be gathered, processed and transported across the United States or other countries throughout the world. A typical engine-driven gas compression package consists of three main components, the first being the skid. The skid is the framework on which all other components of the natural gas compression package are mounted. The skid is typically made of welded structural steel and is custom designed and manufactured for each model of compressor package. The second component is the “driver,” an engine or electric motor. The engine is most commonly a natural gas fired reciprocating internal combustion engine (RICE), ranging from four (4) to 16 cylinders and 100 horsepower to over 5,000 horsepower. In size comparison, the RICE engines are considerably larger to those found in typical automobiles. The last main component of a compressor package is the compressor. Typically, these compressors are reciprocating compressors (RECIP) with at least two (2) large cylinders. Most large RECIP compressors have at least 6 cylinders that are approximately 24 inches in diameter each.

A typical engine-driven compressor package can be broken down into five major lube systems. These systems are:

1) engine lubricating system;

2) engine oil replenishment system;

3) compressor cylinder lube injector system;

4) compressor lubricating system; and

5) new oil bulk tank, or reservoir.

### Engine Lubrication System

The engine oil resides in the engine oil sump. Located at the bottom of the crankcase, the engine oil level must be precisely controlled to ensure that there is both: 1) sufficient oil volume in the sump to provide adequate lubrication; and 2) that there is not excess oil in the sump which would lead to contact between the oil surface and the rotating crankshaft. An over-filled oil sump results in oil ‘foaming’, and power loss due to drag between the oil and the rotating crankshaft.

The engine oil level is traditionally controlled by a float valve in an oil level controller, or maintainer. This maintainer valve is supplied new oil by gravity from an elevated fresh (virgin) bulk oil tank located in close proximity and external to the engine. The maintainer valve opens on demand when the oil level in the sump drops ever so minimally; correspondingly, the valve closes when a sufficient volume of new oil is transferred from the bulk tank to the sump, returning the sump level to normal level.

This engine-maintainer configuration is very vulnerable to installation errors, float valve malfunctions, venting issues between the oil sump and maintainer valve body, and oil line blockages. The engine will shut down automatically by the engine controller module (ECM) if the oil level in the crankcase drops by more than % of an inch. This LOW oil condition could be due to one or more scenarios such as:

1) a leak in the oil supply pipe from the bulk oil tank;

2) a malfunction due to improper installation of the float valve (maintainer);

3) oil consumption due to normal engine operation; and

4) oil bleeding due to an “oil sweetening” system.

On the other hand, the engine will NOT automatically shut down if the oil level in the sump is too high, that is, over-filled. As a result, any improper operation of the oil level control system will either result in unplanned shutdowns (LOW oil condition) or massive engine damage due to over filling the sump with oil. The various types of engine damage can include, but is not limited to: 1) excess oil entering the combustion chamber from the underside of the pistons through the rings, 2) stuck, or ‘seized’ rings due to excess oil entering from the underside of the pistons, caking onto the hot rings, thus freezing up the rings and allowing ‘blow-by’ of combustion gasses, loss of compression, and excess oil entering the combustion chamber leaving behind an abnormal amount of ash on the combustion chamber surfaces.

Internal combustion engines, by their very nature, consume a small amount of oil during the course of normal operation. This oil consumption is primarily the result of the oil lubricating cylinder walls and valve guides within the engine. During each engine cycle, some of the oil is swept up the cylinder walls and between the valve stems and the valve guides. This oil eventually ends up in the combustion chamber where it is burned along with the air and fuel mixture. If the engine oil is too thick (high viscosity) not enough oil makes it onto the cylinder walls or to the valve guides (and is consequently consumed). Aggressive accelerated wear will occur on the metal-to-metal contact surfaces, prematurely aging the engine, and minimizing the interval between overhauls.

Alternatively, if the clearances between the cylinder walls-piston rings, or valve guides-valve stems are excessively large due to improper assembly or advanced engine wear, oil consumption will be increased. Extra oil consumption in the engine creates heavy ash deposits (residue from oil additives) in the combustion chambers, coating the tops of the pistons and valve faces. This ash periodically breaks off and is scraped between the cylinder walls and the piston rings, further accelerating wear and introducing abrasive ash particles into the recirculating oil, thus contaminating the entire engine lubrication system with tiny (sub-micron, >4 micron), abrasive particles.

Embodiments of the inventions, described more thoroughly subsequently, are able to remove much of the risk associated with relying on an automated fault-prone oil level control system to manage the amount of oil in an engine's oil sump. Please understand that the entire disclosure of the U.S. patent application Ser. No. 14/877,896, filed Oct. 7, 2015, is incorporated into this document by reference. In other embodiments of the inventions, the oil consumption monitoring device is able to precisely measure extremely small volumes of oil that enters the sump in real-time. In still other embodiments of the inventions, the oil flow data is processed “locally” by a microprocessor which is mounted on the engine and combined with timestamp information (both calendar time and machine operating time). In yet other embodiments of the inventions, the oil flow data processed by the microprocessor is then transmitted over the internet to a database server where it is cataloged and sent to the application server for further data analytic processing. The remote application server processes inputs from the remote field flow measuring devices into “actionable information.”

In order to accomplish this, the application server executes custom developed compute statistic algorithms, in embodiments of the inventions, such as:

1) flow rate averaging;

2) oil level averaging;

3) differential pressure averaging;

4) event identification based on high/low flow rates/oil levels/differential pressures;

5) event identification based on based a high volume over a given time;

6) averaging oil flow based on the fraction of time the machine was in operation (instead of over the entire time oil flow was measured); and

7) predictive trending (simultaneously over time periods ranging from minutes to years) of flow rates to allow for automatic detection of abnormal conditions.

Lastly, real-time oil flow comparison between similar machines in a compression package equipment fleet enables the abnormal behavior of one machine to be more easily detected. From the processed data, the application server executes and computes statistic algorithms that are able to identify the existence of “dangerous machine conditions” such as LOW oil flow, NO oil flow, or EXCESSIVE oil flow, and then immediately initiate and appropriate alarm notification accordingly.

### Engine Oil Replenishment System

The engine oil replenishment (sweetening) system continuously bleeds off, or removes, a precise amount of oil from the engine's oil sump so that the old oil can be replaced with fresh oil as supplied via gravity from an on-site bulk oil tank. The rate of oil replacement is automatically managed by the engine-mounted oil sump level control system mentioned above. This oil replenishment system must be optimized so that the proper volume of engine oil is removed in a given day to maintain the desired engine oil cleanliness levels but not so much that the oil is being wasted by being prematurely removed from the engine before it reaches the end of its useful life. The oil replenishment rate is generally derived from oil change interval recommendations made by the original equipment manufacturer. Additionally, the U.S. Environmental Protection Agency (EPA) has enacted a law (RICE NESHAP) that regulates the oil change interval. A popular strategy developed by compression package equipment operators is to adopt an engine oil sweetening system that bleeds off the engine oil at a rate that matches the OEM manufacture recommendations, thus complying with the RICE NESHAP regulations.

Embodiments of the invention help customers to optimize and refine their oil sweetening process by using the real-time oil flow rate measurement, coupled with additional machine oil quality attributes from the data server (for example, Total Acid Number, Total Base Number). The oil replenishment rates can be adjusted automatically on the fly to perfectly balance desired oil cleanliness against the economic savings related to minimizing the oil replenishment rate. Too fast a replenishment rate is costly to the operator. Too low of a replenishment rate could potentially subject the engine to operating with a poor (i.e. low) oil quality, thus damaging the engine.

### Compressor Cylinder Lube Injector System

The compressor cylinder lube injector system is designed to provide lubrication in the form of oil to the compressor cylinders and gas flow valves. The source of the cylinder injector oil can be: 1) dedicated cylinder lubrication injector oil (different than compressor crankcase oil), 2) oil diverted, or bled, from the engine crankcase sump by a replenishment (sweetening) system, or 3) oil diverted, or bled, from the compressor crankcase sump by a replenishment (sweetening) system. The natural gas product being compressed possesses almost no lubricating qualities so supplemental lubricant is injected into the compressor cylinder to reduce friction between the cylinder walls and pistons, thus extending intervals between overhauls, increasing overall compressor life, increasing the system uptime, and reducing operating costs.

In the event of a compressor cylinder lube injection system failure, the compressor will run without the supplemental lubricating oil provided by the injection system. As a result, the compressor will become very hot and inefficient. In the worst case scenario, the one-way valves that allow the compressor to pump natural gas will seize shut from the excess heat and require a complete rebuild to repair the problem. In the best case scenario, the compressor cylinder lube injection system will be equipped with a simple battery operated no-flow alarm that automatically shuts down the compressor package in the event of a compressor cylinder lube injector failure.

Each compressor configuration has an ideal cylinder injector lubrication rate that is recommended by the OEM manufacturer. Embodiments of the inventions provide the operator with real-time actionable information related to the oil consumption, or lubrication, rate. The target lubrication rate (advised by the OEM) can be entered into the Dangerous Condition threshold settings of the invention's microprocessor. If a “dangerous condition” condition is determined, that is, a lubrication rate threshold has been violated, the invention can automatically generate an alarm notification that informs the operator of the dangerous condition.

Over-lubrication of the compressor cylinders will send carryover oil downstream in the pipeline, thus contaminating the product gas resulting in a lower quality gas product. Worst case scenario is if the gas contains an excessive amount of suspended oil that is detected at the custody transfer point and is deemed unsaleable. Other downside of this carryover oil is the need to ‘pig’ the pipeline to remove residual oil and any varnish build up. Under-lubrication can result in excess friction in the compressor cylinders, generating excess heat and accelerated wear; a shortened interval between overhauls would likely result. Embodiments of the inventions can provide the operator with advanced warning and notification of either an under-lubrication and over-lubricating condition.

The lubricating oil used by the compressor cylinder lube injector system is filtered prior to being sent to the compressor high pressure lube pump where it is pressurized to extremely high pressures (over 1000 psi) and forced through individual injector nozzles located in the cylinder heads of the compressor. This oil filter is responsible for removing any abrasive particles or other contaminates that could potentially damage the compressor high pressure lube pump, lube injectors, the compressor cylinder walls and piston rings. If the filter fails to remove abrasive particles from the oil (e.g. ash particles, residual from oil consumed in the combustion chamber), it is likely that the compressor lube injection system, the compressor cylinder and gas valves will be subjected to highly accelerated abrasive wear.

The invention is able to solve the problems associated with compressor cylinder lube injector failure by predictively trending the performance of the lube injectors over time. In this way, advance warning can be given to the operator before a compressor cylinder lube injector system experiences a failure. This advance warning from the invention allows customers to preventatively service the compressor lube injector system at their convenience, when their schedule allows.

Since the compressor cylinder lube injection systems are very sensitive to small abrasive particles, it is very important to manage the status of the filter installed to protect the lube injection system from excessive wear. If the lube injection system filter becomes loaded (filled) with abrasive particles, thereby clogging the filter, it will restrict the flow of oil to the compressor cylinder lube injection system. Alternatively, if the lube injection filter is malfunctioning, or insufficiently capable of capturing the abrasive particles, it will never show an increase in differential pressure (e.g. increasing flow restriction), thus permitting abrasive particles to pass through the filter media and enter into the compressor cylinder lube injection system. The invention, in the form of a novel differential pressure meter device, is able to apply the same data processing and trending algorithms (invention) as mentioned above to the measured filter differential pressure (flow restriction) data and alert operators to future compressor cylinder lube injection system failures.

### Compressor Lubricating System

Just as the engine possesses a lubricating oil sump in the crankcase, the compressor also possesses a lubricating oil sump in its respective crankcase that contains oil used to lubricate the crankcase, connecting rods and block. The compressor is also equipped with an oil level control system, as is found on the engine. However, unlike the engine, consumptive oil loss from the compressor is very small. In fact, oil consumption in a reciprocating compressor is an indicator of a potential problem that is either wear related or indicative of a major compressor piston or piston ring failure.

The invention allows for the compressor oil consumption rate to be precisely measured in real-time, down to extremely small volumes of replenishment oil entering the sump. The oil flow data is processed “locally” by a microprocessor (integral to the system) which is mounted on the engine and combined with timestamp information (both calendar time and machine operating time). The oil flow data processed by the microprocessor is then transmitted over the internet to a database server where it is cataloged and sent to the application server for further data analytic processing. The remote application server processes inputs from the remote field flow measuring devices into “actionable information.”

In order to accomplish this, the application server executes custom developed compute statistic algorithms (invention) such as:

1) flow rate averaging;

2) oil level averaging;

3) differential pressure averaging;

4) event identification based on high/low flow rates/oil levels/differential pressures;

5) event identification based on based a high volume over a given time;

6) averaging oil flow based on the fraction of time the machine was in operation (instead of over the entire time oil flow was measured); and

7) predictive trending (simultaneously over time periods ranging from minutes to years) of flow rates to allow for automatic detection of abnormal conditions.

Lastly, real-time oil flow comparison between similar machines in a compression package equipment fleet enables the abnormal behavior of one machine to be more easily detected. From the processed data, the application server executes compute statistic algorithms that are able to identify the existence of “dangerous machine conditions” such as LOW oil flow, NO oil flow, or EXCESSIVE oil flow, and then immediately initiate an appropriate alarm notification accordingly.

### New Oil Bulk Tank

New (virgin) oil is stored on site in a large bulk oil tank, sometimes referred to as a ‘day tank’, or reservoir. This tank is typically mounted in an elevated position so that it can use a gravity feed to provide oil to the:

1) engine oil sump;
2) compressor oil sump; and
3) compressor cylinder lube injection system.

Traditionally, the bulk oil tank level is checked by the customer via a tank-mounted sight glass that allows for the manual visual observation of the approximate tank level. In the event of an oil leak or excessive oil consumption, the tank can be emptied (without knowledge of the operator), potentially depriving the equipment of the required oil supply. The bulk oil tank must also be manually filled by the bulk oil distributor, requiring the customer or the distributor to carefully manage the tank level. In the event that the bulk oil tank level is improperly managed, the oil tank may not be filled as often as is really required. This will also lead to the tank being inadvertently emptied earlier than expected.

Embodiments of the inventions are able to solve the problems associated with managing the oil level in a bulk oil tank by combining the real-time flow rate measurements from all of the flow measurement devices and comparing these flow rates against the rate of change of the bulk tank level sensing device. This allows for the oil flowing out of the bulk tank to be “accounted” for against all of the oil flow rates measured by the oil flow measurement devices. If all of the flow rates do not add up to zero, it is likely that a leak is present in the oil plumbing system. The embodiments of the inventions will provide the customer with a “dangerous condition” alert that a leak is present so that it can be remedied before the bulk oil tank is drained completely. Additionally, the oil level measurement device allows for intelligent scheduling of oil deliveries as the inventions can track the rate of oil depletion from the tank and predictively forecast the date and time that the tank will be empty, or need to be refilled. The customer will no longer be required to manually and visually check the oil level within the bulk oil tank. As a result, oil suppliers and customers can both make better use of their time as the invention will keep track of oil levels and make the information conveniently available via a web-based dashboard or an interactive mobile application for handheld devices (for example, smartphones).

### Cooling System Flow Monitoring

Compressor packages utilize antifreeze coolant (glycol/water mixture) by circulating it through the engine/compressor and then through a heat exchanger to maintain the equipment at safe operating temperatures. Coolant leaks (that would minimally result in environmental cleanup fines and maximally result in total catastrophic failure of the engine and/or compressor) can be quickly detected and repaired using the flow measurement invention.

The inventive flow measurement devices/systems, coupled with the inventive liquid level measurement devices/systems, can measure the coolant circulating through the engine or compressor and the amount of coolant installed in the machine. This enables users to take account of all of the coolant entering and leaving the engine or compressor. If the flow rates into and out of the engine/compressor become significantly different, the invention may indicate the presence of:
1) Coolant (i.e. water/glycol mixture, cooling oil, etc. . . . ) leak external to the machine, may lead to excessive coolant loss, overheating the machine, environmental clean-up costs/fines associated with a spill.
2) Coolant leak internal to the machine, fast detection of such a leak may prevent catastrophic engine failure or help to avoid extensive damage and downtime.
3) Plugged or clogged coolant passages within the machine or the radiator. The existence of which will lead to reduced cooling efficiency, higher operating temperatures, and increased likelihood of overheating.
4) Early warnings of coolant pump failure.

### Compressor Package Engine Crankcase Ventilation System Flow Measurement

Crankcase ventilation system flow measurement is also possible with the inventive flow measurement device/system. By measuring the flow of gasses into and out of a machine's crankcase in real-time, the machine can be more effectively monitored for potentially harmful operating conditions. Since the crankcase ventilation system is designed to constantly replenish the crankcase gasses (oil vapors, fuel vapors, combustion gasses, soot and other combustion byproducts), low positive crankcase ventilation system (PCV) flow conditions can be caused by clogged PCV filters, obstructed ventilation pipes, or improper engine pressures and will result in:

1) excessive oil contamination;

2) oil thinning; and

3) accelerated wear on the machine.

High PCV system flow conditions are indicators of potentially severe machine conditions such as:

1) Excessive cylinder/piston ring wear that leads to high amounts of blow-by. This indicates that the engine is no longer operating at peak power and efficiency. It also means the engine is blasting the crankshaft and oil sump with corrosive, high temperature combustion gasses. This results in high crankcase temperatures which increases rates of oil oxidation and varnish creation, shortening the oil life and further accelerating engine wear.

2) Damaged, cracked, or compromised piston. If a piston develops a crack or hole, it will allow large amounts of air, combustion byproducts, and unburned fuel into the crankcase. Much like the above case, this means that the engine is not running properly, the oil is being artificially degraded, and there is the possibility of a catastrophic piston failure which will result in large amounts of downtime and a potential complete engine overhaul.

### Fuel Monitoring

The inventive flow level and pressure measurement devices/systems are equally capable of measuring, processing, and displaying data relating to all liquid or gas (vaporous), petroleum fuel and fuel processing systems that may be found in industrial, residential, or commercial applications. All features such as processing data related to flow rate, consumption, leak detection, real time display, dangerous condition detection and predictive analysis on the collected data can be performed on liquid or gas (vaporous) petroleum fuel systems.

### Potable Water Monitoring

The inventive flow level and pressure measurement devices/systems are equally capable of measuring, processing, and displaying data relating to potable (fresh) water systems that may be found in industrial, residential, or commercial applications. All features such as processing data related to flow rate, consumption, leak detection, real time display, dangerous condition detection and predictive analysis on the collected data can be performed on potable water systems.

### Water Monitoring/Water Based Solution Monitoring

The inventive flow level and pressure measurement devices/systems are equally capable of measuring, processing, and displaying data relating to water systems that contain either non-potable water (e.g. grey water) or aqueous solutions which can be (but are not required to be) composed of multi-phase flows and/or precipitates (i.e. glycol/water mixtures, manufacturing processes, cleaning processes, etc.) the inventions are applicable is most industrial, residential, or commercial systems that utilize water or water based solutions. All features such as processing data related to flow rate, real time display, consumption, leak detection, dangerous condition detection and predictive analysis on the collected data can be performed on such water systems.

Feedback Control Inputs from External Data

As mentioned previously, the inventive liquid flow rate, level, and pressure measurement inventions are capable of accepting control inputs as the result of feedback from external data feeds (i.e. fluid quality, qualitative/quantitative analysis data, manual operator input, etc.). Such control inputs allow the inventions to adjust parameters such as flow rate or liquid level in order to optimize a given mechanical system or other technical process.

### Conclusion

Embodiments of the inventions mentioned above are more thoroughly described below, or are more thoroughly described in the U.S. patent application Ser. No. 14/877,896, filed Oct. 7, 2015, stated previously as being incorporated into this document by reference. The inventions provide customers with a comprehensive and reliable tool for managing the lubricant consumption of their gas compression packages as well as allowing for early detection of potentially serious maintenance needs of the same gas compression package equipment. On-the-fly adjustments to oil replenishment rates and oil delivery schedules can be made based on the real-time condition of the natural gas compression package being measured. The data measured by the invention can be analyzed across an entire fleet of gas compression packages to identify inefficiencies and reliability concerns that would otherwise go unnoticed. The invention provides a myriad of novel and unprecedented tools for managing and optimizing valuable gas compression packages and ensuring that they are always properly lubricated and operating in the most efficient manner possible. When used properly, the invention will easily become the new standard “best practices” solution for machine health based gas compression fleet management.

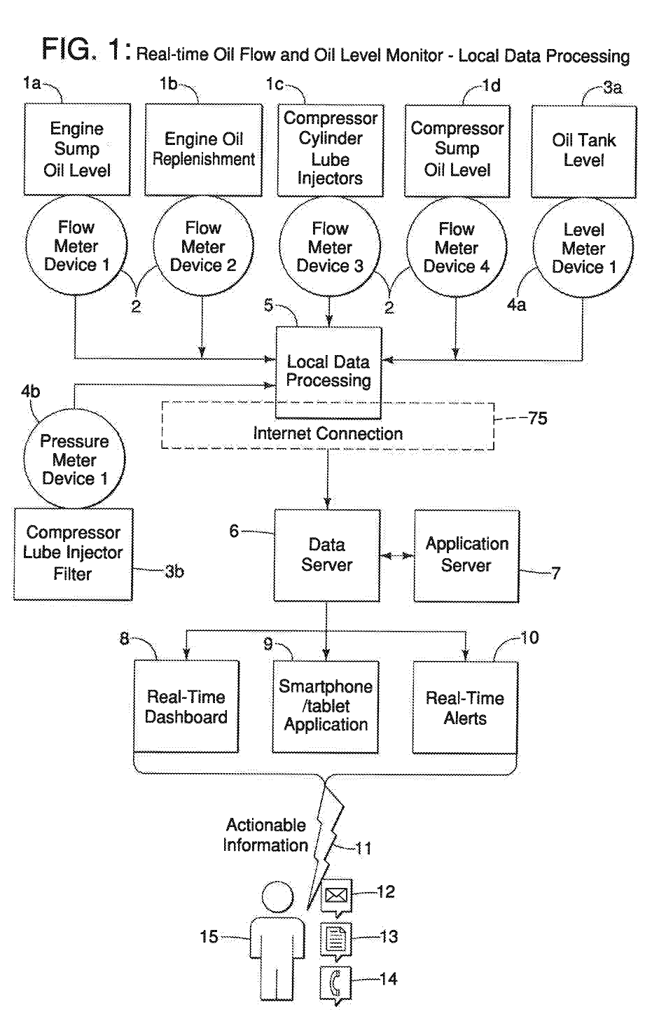

Referring to FIG. 1, an Exemplary LOCAL Data Processing for Real-Time Oil Flow and Oil Level Monitor is Shown.

### Logic Diagram Item Key:

Reference number (hereinafter “Ref. no.”) 1) Equipment being monitored for oil consumption:
a) Internal combustion engine, crankcase oil sump level, measured or derived value;
b) Internal combustion engine oil consumption rate, rate of oil replenishment, measured or derived value;
c) Compressor cylinder lubrication oil injection rate, measured or derived value;
d) Compressor or other driven device, crankcase oil sump level, rate of oil replenishment, measured or derived value;
e) Compressor or other driven device crankcase oil consumption rate, measured or derived value [not shown];
f) Gearbox oil sump level, rate of oil replenishment, measured or derived value [not shown];
g) Electric motor bearing oil consumption rate, measured or derived value [not shown];
h) Turbine oil sump level, rate of oil replenishment, measured or derived value [not shown]; and
i) Other machinery utilizing a lubrication system with integrated lubricant replenishment [not shown].

Ref no. 2) Novel oil flow measurement device (also fluid flow monitoring and management device or flow meter device) (four shown but could be more or less (1-3 and 4- . . . )). Example: a positive displacement gear meter with a direct current (DC) pulse output; the output pulses correlating with a known fluid volume per each full gear revolution, or corresponding pulse. A pulse signal sent to local processing unit (5) for data flow rate computations.

Ref no. 3) Equipment being monitored for oil consumption:
a) Reservoir tank(s) containing lubricant (oil). Provides make-up oil for replenishment of oil consumed by machinery functions, and or leaks.

Further regarding the tank marked as 3a, such tank can be additional termed a “Make Up Oil Tank”. It should be understood that the terms “make up” refers to replenishment of the oil that is consumed by the engine and/or the compressor. This tank is also referred to as a “day tank” or “reservoir”.

It should be further understood that a separate Make Up Oil Tank (3a) may be dedicated to an engine or a compressor, or in one embodiment, one (1) Make Up Oil Tank can be used for BOTH an engine and compressor (in the case of a compressor/engine package typical of natural gas gathering/processing applications where the SAME lube oil grade is used for both engine and compressor. In other embodiments, the engine lube oil is different than the compressor lube oil. Hence, each system will need a separate Make Up Oil Tank (3a).
b) Filter for compressor cylinder lube injector system. Removes contaminates from oil before it enters the lube injector pump and compressor divider block lubrication injectors.

Ref no. 4) Novel fluid level (4a) and pressure measurement devices (4b).
a) Fluid level sensor sending unit, i.e. pressure transducer, etc.
b) Differential pressure (DP) sensor sending unit, i.e. pressure transducer, etc.

Ref no. 5) Local data processing unit, i.e. microcontroller (invention) having specific features, such as the ability to measure and totalize the meter inputs (i.e. pulses, analog voltages, analog currents, and digital signals), the ability to detect if the machine is in an ON or OFF state, a real-time-clock (RTC) unit for timekeeping, communication to the internet, either wired or wireless. The local data processing unit is also capable of removing false alarms that come from temporary sensor or measurement errors.

Ref no. 75) Internet connection from local data processing unit (5) to the data server, which can be situated either remotely or locally, to the measurement device(s). The connection to the internet can be direct (e.g. built in cellular modem) or indirect (e.g. serial/WiFi/Ethernet link to a modem).

Ref no. 6) Data server (remote or local) having specific features such as the ability to store and organize the collected flow/level/differential pressure data with respect to calendar time and machine operating time.

Ref no. 7) Application server processes inputs into “actionable information”, executes custom developed compute statistics algorithms (i.e. flow rate/level/differential pressure averaging, event identification) based on high/low flow rates/levels/differential pressures or based on a high volume over a given time; averaging oil flow based on the fraction of time the machine was in operation (instead of over the entire elapsed time the oil flow was measured); and predictive trending (simultaneously over time periods ranging from minutes to years) of flow rates to allow for automatic detection of abnormal conditions. Lastly, real-time flow comparison between similar machines in a fleet enables the abnormal behavior of one machine to be more easily detected. Compute statistics algorithms are able to identify the existence of dangerous machine conditions (Dangerous Conditions) and alarm accordingly.

Ref no. 8) Results output—dashboard, i.e. web-based dashboard displaying real-time actionable information. Results output also promotes fleet-wide diagnostics and optimization.

Ref no. 9) Mobile application (invention) running on hand-held device, i.e. smartphone, tablet, etc. displaying real-time information, provides users with the ability to configure system settings.

Ref no. 10) System thresholds and alert notification settings and algorithm inputs for determining Dangerous Conditions, and exception-based reporting.

Ref no. 11) Real-time actionable information is transmitted to user via telephone or internet connection.

Ref no. 12) User communication, i.e. alert notifications, system status, real-time actionable information, sent via email.

Ref. no. 13) User communication, i.e. alert notifications, system status, real-time actionable information, sent via SMS text message.

Ref no. 14) User communication, i.e. alert notifications, system status, real-time actionable information, sent via automated phone call.

Ref no. 15) User of the Novel Real-time Oil Flow and Oil Level Monitor system (invention) receives clear and actionable information on engine and compressor operation.

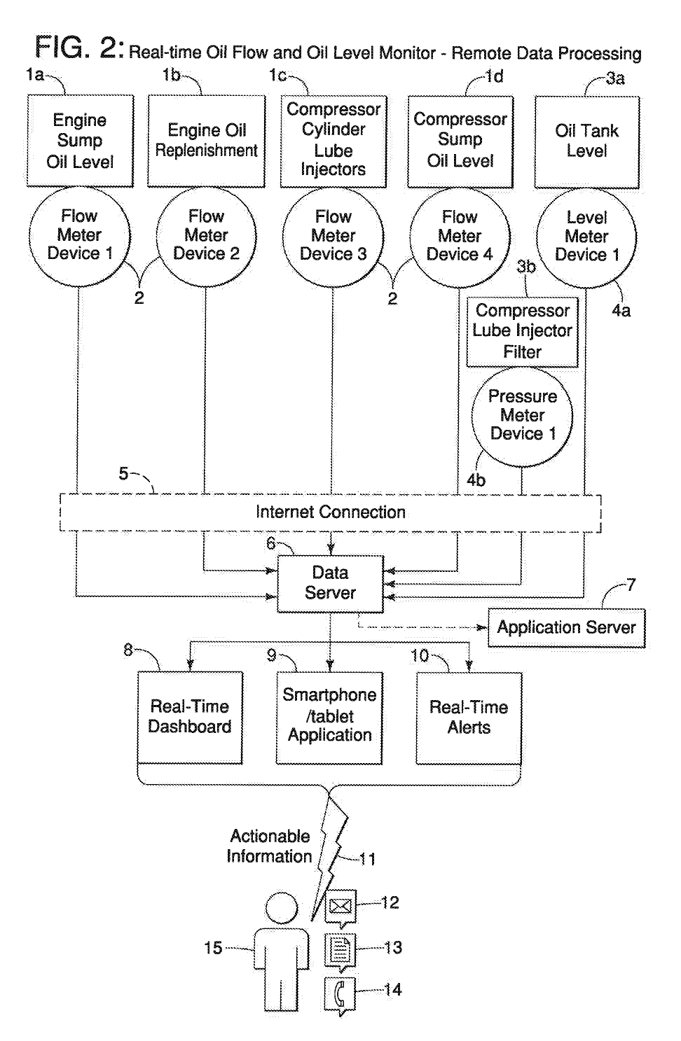

Referring to FIG. 2, an Exemplary REMOTE Data Processing for Real-Time Oil Flow and Oil Level Monitor is Shown.

Logic Diagram Item Key (hereinafter, “Ref. no.” not presented before reference numbers):

1) Equipment being monitored for oil consumption:
a) Internal combustion engine, crankcase oil sump level, measured or derived value
b) Internal combustion engine oil consumption rate, rate of oil replenishment, measured or derived value
c) Compressor cylinder lubrication oil injection rate, measured or derived value
d) Compressor or other driven device, crankcase oil sump level, rate of oil replenishment, measured or derived value
e) Compressor or other driven device crankcase oil consumption rate, measured or derived value [not shown]
f) Gearbox oil sump level, rate of oil replenishment, measured or derived value [not shown]
g) Electric motor bearing oil consumption rate, measured or derived value [not shown]
h) Turbine oil sump level, rate of oil replenishment, measured or derived value [not shown]
i) Other machinery utilizing a lubrication system with integrated lubricant replenishment [not shown]
   2) Novel oil flow measurement device (invention). Example: a positive displacement gear meter with a direct current (DC) pulse output; the output pulses correlating with a known fluid volume per each full gear revolution, or corresponding pulse. A pulse signal sent to local processing unit (5) for data flow rate computations.
   3) Equipment being monitored for oil consumption:
a) Reservoir tank(s) containing lubricant (oil); provides make-up oil for replenishment of oil consumed by machinery functions, and or leaks.
b) Filter for compressor cylinder lube injector system. Removes contaminates from oil before it enters the lube injector pump and compressor divider block lubrication injectors.
   4) Novel fluid level and pressure measurement devices (invention).
a) Fluid level sensor sending unit, i.e. pressure transducer, etc.
b) Differential pressure (DP) sensor sending unit, i.e. pressure transducer, etc.
   5) Internet connection from novel flow, level, or differential pressure metering device (invention) to the data server, which can be situated either remotely or locally, to the measurement device(s). The connection to the internet can be direct (e.g. built in cellular modem) or indirect (e.g. serial/WiFi/Ethernet link to a modem).
   6) Data server, remote or local, having specific features such as the ability to store and organize the collected flow/level/differential pressure data with respect to calendar time and machine operating time. The data server assigns required attributes such as timestamps to the data as it is received from the field devices.
   7) Application server processes inputs into actionable information, executes custom developed compute statistics algorithms (i.e. flow rate/level/differential pressure averaging, event identification) based on high/low flow rates/levels/differential pressures or based on a high volume over a given time; averaging oil flow based on the fraction of time the machine was in operation (instead of over the entire elapsed time the oil flow was measured); and predictive trending (simultaneously over time periods ranging from minutes to years) of flow rates to allow for automatic detection of abnormal conditions. Lastly, real-time flow comparison between similar machines in a fleet enables the abnormal behavior of one machine to be more easily detected. Compute statistics algorithms are able to identify the existence of dangerous machine conditions (Dangerous Conditions) and alarm accordingly.
   8) Results output—dashboard (e.g. web-based dashboard) displaying real-time actionable information. Results output also promotes fleet-wide diagnostics and optimization.
   9) Mobile application (invention) running on hand-held device, i.e. smartphone, tablet, etc. displaying real-time information, provides users with the ability to configure system settings.
   10) System thresholds and alert notification settings and algorithm inputs for determining Dangerous Conditions, and exception-based reporting.
   11) Real-time actionable information is transmitted to user via telephone or internet connection.
   12) User communication, i.e. alert notifications, system status, real-time actionable information, sent via email.
   13) User communication, i.e. alert notifications, system status, real-time actionable information, sent via SMS text message.
   14) User communication, i.e. alert notifications, system status, real-time actionable information, sent via automated phone call.
   15) User of the Novel Real-time Oil Flow and Oil Level Monitor system (invention) receives clear and actionable information on engine and compressor operation.

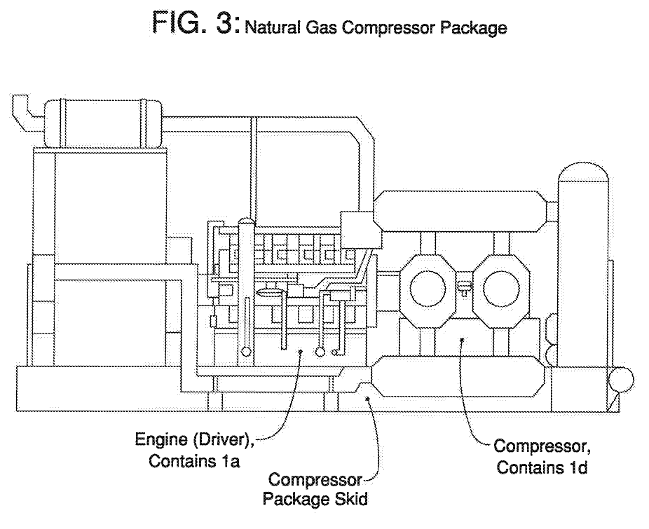

Referring to FIG. 3, an Exemplary Natural Gas Compressor Package is Shown.

### Logic Diagram Item Key:

1) Equipment being monitored for oil consumption:
a) Internal combustion engine, crankcase oil sump level, measured or derived value
b) Internal combustion engine oil consumption rate, rate of oil replenishment, measured or derived value [not shown]
c) Compressor cylinder lubrication oil injection rate, measured or derived value [not shown]
d) Compressor or other driven device, crankcase oil sump level, rate of oil replenishment, measured or derived value

### Figure Details:

FIG. 3 represents the typical configuration of a natural gas compression package. An engine driven gas compressor package is a device that is used very commonly in the oil and gas industry to “pump, or compress” natural gas so that it can be gathered, processed and transported across the United States or other countries throughout the world. A typical engine driven gas compression package consists of three main parts, the first being the Skid. The skid is the framework that all other components of the natural gas compression package is mounted to. It is typically made of welded structural steel and is custom designed and manufactured for each model of compressor package. The second is the Engine, or driver. The engine is most commonly a natural gas powered fired reciprocating internal combustion engine (RICE), ranging from 4 to 16 cylinders and 100 horsepower to over 5,000 horsepower. These RICE engines are very similar to those found in typical automobiles, although they are much larger. The last main component of a compressor package is the compressor. Typically, these compressors are reciprocating compressors (RECIP) with at least 2 large cylinders. Most large compressors have at least 6 cylinders that are approximately 24 inches in diameter each.

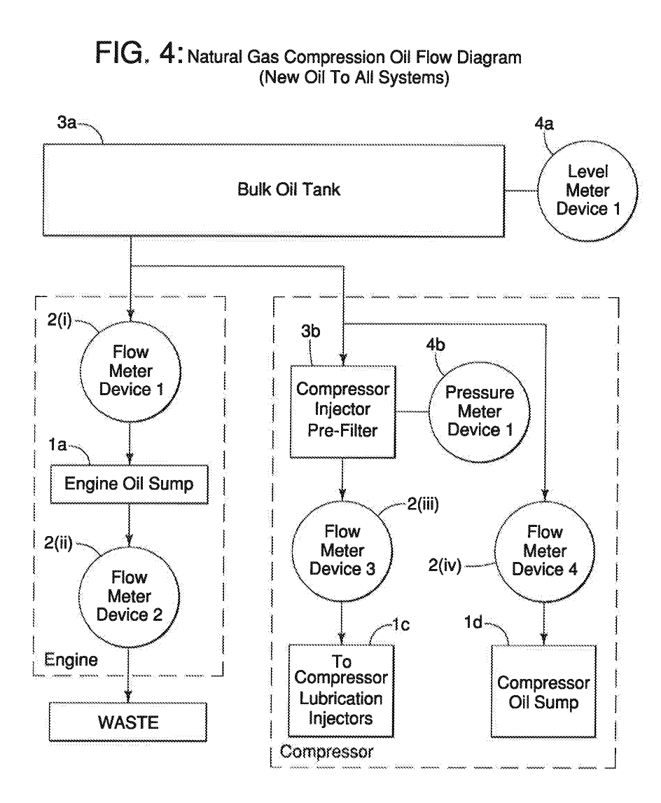

Referring to FIG. 4, an Exemplary Natural Gas Compression Package Oil Flow Diagram is Shown. This Exemplary Embodiment Illustrates New Oil to all Systems.

### Logic Diagram Item Key:

1) Equipment being monitored for oil consumption:
a) Internal combustion engine, crankcase oil sump level, measured or derived value;
b) Internal combustion engine oil consumption rate, rate of oil replenishment, measured or derived value;
c) Compressor cylinder lubrication oil injection rate, measured or derived value; and
d) Compressor or other driven device, crankcase oil sump level, rate of oil replenishment, measured or derived value
   2) Novel oil flow measurement devices (2(i, ii, iii, iv)). Example: a positive displacement gear meter with a direct current (DC) pulse output; the output pulses correlating with a known fluid volume per each full gear revolution, or corresponding pulse. A pulse signal sent to local processing unit (5) for data flow rate computations.
   3) Equipment being monitored for oil consumption:
a) Reservoir tank(s) containing lubricant (oil). Provides make-up oil for replenishment of oil consumed by machinery functions, and or leaks.
b) Filter for compressor cylinder lube injector system. Removes contaminates from oil before it enters the lube injector pump and compressor divider block lubrication injectors.
   4) Novel fluid level and pressure measurement devices (invention):
a) Fluid level sensor sending unit (level meter device), i.e. pressure transducer, etc.
b) Differential pressure (DP) sensor sending unit (pressure meter device), i.e. pressure transducer, etc.

### Oil Flow Description:

1) New oil is supplied directly from the bulk oil tank to the:
a) engine oil sump
b) compressor oil sump
c) compressor cylinder lube injection system.
   2) Flow meter devices (2(i, iii, iv)) are located on the inlets of the engine oil sump, compressor oil sump and the compressor cylinder lube injection system to measure the flow entering each system.
   3) A flow meter device (2(ii)) is also located on the exit of the engine oil sump to measure the oil replenishment rate (flow of oil leaving the engine oil sump).
   4) The numerical difference between the flow of oil entering the engine oil sump and the replenishment flow (sweetening) rate is calculated to be the rate of oil consumption within the engine.
   5) A pressure meter device is used to measure the flow restriction provided by the compressor lube injector pre-filter, and can be used to detect a filter malfunction or indicate that the filter must be changed.
   6) A level meter device (invention) is installed on the bulk oil tank to measure:
a) the oil level in the bulk oil tank
b) the flow rate of oil out of the bulk oil tank
   7) Leaks in the system can be detected if the flow rate out of the bulk oil tank is measured to exceed the sum of the flow rates into the engine oil sump, compressor oil sump and the compressor cylinder lube injection system.

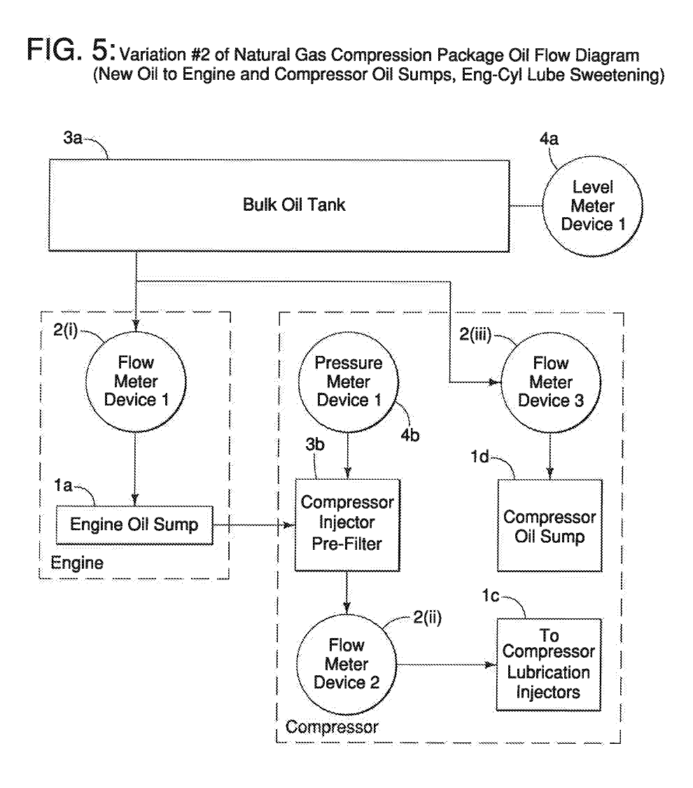

Referring to FIG. 5, and Exemplary Natural Gas Compression Package Oil Flow Diagram is Shown (in this Exemplary Embodiment, New Oil is Provided to Engine and Compressor Oil Sumps, Engine-Cylinder Lube Sweetening).

### Logic Diagram Item Key:

1) Equipment being monitored for oil consumption:
a) Internal combustion engine, crankcase oil sump level, measured or derived value;
b) Internal combustion engine oil consumption rate, rate of oil replenishment, measured or derived value;
c) Compressor cylinder lubrication oil injection rate, measured or derived value;
d) Compressor or other driven device, crankcase oil sump level, rate of oil replenishment, measured or derived value;
e) Compressor or other driven device crankcase oil consumption rate, measured or derived value [not shown];
f) Gearbox oil sump level, rate of oil replenishment, measured or derived value [not shown];
g) Electric motor bearing oil consumption rate, measured or derived value [not shown];
h) Turbine oil sump level, rate of oil replenishment, measured or derived value [not shown]; and
i) Other machinery utilizing a lubrication system with integrated lubricant replenishment [not shown].
   2) Novel oil flow measurement device (invention). Example: a positive displacement gear meter with a direct current (DC) pulse output; the output pulses correlating with a known fluid volume per each full gear revolution, or corresponding pulse. A pulse signal sent to local processing unit (5) for data flow rate computations.
   3) Equipment being monitored for oil consumption:
a) Reservoir tank(s) containing lubricant (oil). Provides make-up oil for replenishment of oil consumed by machinery functions, and or leaks.
b) Filter for compressor cylinder lube injector system. Removes contaminates from oil before it enters the lube injector pump and compressor divider block lubrication injectors.
   4) Novel fluid level and pressure measurement devices.
a) Fluid level sensor sending unit, i.e. pressure transducer, etc.
b) Differential pressure (DP) sensor sending unit, i.e. pressure transducer, etc.

### Oil Flow Description:

1) New oil is supplied directly from the bulk oil tank to the:
a) engine oil sump
b) compressor oil sump
   2) Used oil exits the engine oil sump and is redirected (pumped under pressure) to the compressor cylinder lube injection system, first passing through an in-line oil filter to remove contaminates from the engine oil.
   3) The engine oil sump level is maintained by the engine oil replenishment system, i.e. oil sump level controller, maintainer, etc.
   4) Flow meter devices (invention) are located on the inlets of the engine oil sump, compressor oil sump and the compressor cylinder lube injection system to measure the flow entering each system.
   5) The difference between the flow of oil entering the engine oil sump and the replenishment flow rate (flow of oil leaving the engine oil sump) is calculated to be the rate of oil consumption within the engine.
   6) The flow meter device which is located between the exit of the engine oil sump and the inlet of the compressor cylinder lube injection system measures both the oil replenishment rate and the compressor lube injection system flow rate.
   7) A pressure meter device is used to measure the flow restriction provided by the compressor lube injector pre filter, and can be used to detect a filter malfunction or indicate that the filter must be changed. This is very critical in this case, since the lube injection system is being supplied with used (dirty) engine oil.
   8) A level meter device is installed on the bulk oil tank to measure:
a) the oil level in the bulk oil tank
b) the flow rate of oil out of the bulk oil tank
   9) Leaks in the system can be detected if the flow rate out of the bulk oil tank is measured to exceed the sum of the flow rates into the engine oil sump, compressor oil sump and the compressor cylinder lube injection system.

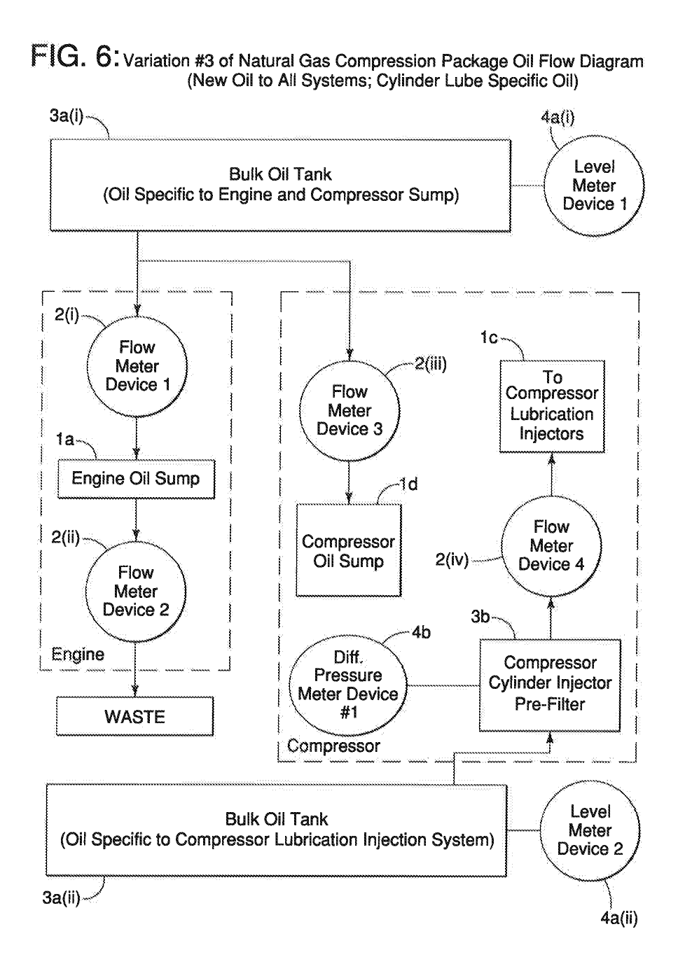

Referring to FIG. 6, an Exemplary Natural Gas Compression Package Oil Flow Diagram is Shown (in this Exemplary Embodiment, New Oil to all Systems; Cylinder Lube Specific Oil).

### Logic Flow Diagram Item Key:

1) Equipment being monitored for oil consumption:
a) Internal combustion engine, crankcase oil sump level, measured or derived value;
b) Internal combustion engine oil consumption rate, rate of oil replenishment, measured or derived value;
c) Compressor cylinder lubrication oil injection rate, measured or derived value;
d) Compressor or other driven device, crankcase oil sump level, rate of oil replenishment, measured or derived value;
e) Compressor or other driven device crankcase oil consumption rate, measured or derived value [not shown];
f) Gearbox oil sump level, rate of oil replenishment, measured or derived value [not shown];
g) Electric motor bearing oil consumption rate, measured or derived value [not shown];
h) Turbine oil sump level, rate of oil replenishment, measured or derived value [not shown]; and
i) Other machinery utilizing a lubrication system with integrated lubricant replenishment [not shown].
   2) Novel oil flow measurement device. Example: a positive displacement gear meter with a direct current (DC) pulse output; the output pulses correlating with a known fluid volume per each full gear revolution, or corresponding pulse. A pulse signal sent to local processing unit (5) for data flow rate computations.
   3) Equipment being monitored for oil consumption and condition status:
a) Reservoir tank(s) containing lubricant (oil). Provides make-up oil for replenishment of oil consumed by machinery functions, and or leaks.
b) Filter for compressor cylinder lube injector system. Removes contaminates from oil before it enters the lube injector pump and compressor divider block lubrication injectors.
   4) Novel fluid level and pressure measurement devices.
a) Fluid level sensor sending unit, i.e. pressure transducer, etc.
b) Differential pressure (DP) sensor sending unit, i.e. pressure transducer, etc.

### Oil Flow Description:

1) New (virgin) oil is supplied directly from bulk oil tank #1 to the:
a) engine oil sump; and
b) compressor oil sump.
   2) Cylinder lube-specific new oil is supplied directly from bulk oil tank #2 to the lube injection system.
   3) Flow meter devices (invention) are located on the inlets of the:
a) engine oil sump;
b) compressor oil sump; and
c) compressor cylinder lube injection system, to measure the flow entering each system.
   4) A flow meter device (invention) is also located on the exit of the engine oil sump to measure the oil replenishment (sweetening) rate (flow of oil leaving the engine oil sump).
   5) The difference between the flow of oil entering the engine oil sump and the replenishment (sweetening) flow rate is calculated to be the rate of oil consumption within the engine.
   6) A differential pressure meter device is used to measure the flow restriction provided by the compressor lube injector pre filter, and can be used to detect a filter malfunction or indicate that the filter must be changed.
   7) A level meter device (invention) is installed on both bulk oil tank(s) to measure:
a) the oil level in the bulk oil tank(s); and
b) the flow rate of oil out of the bulk oil tank(s).
   8) Leaks in the system can be detected if the flow rate out of the engine/compressor bulk oil tank (tank #1) is measured to exceed the sum of the flow rates into the engine oil sump and the compressor oil sump. The same technique is also applied to the cylinder lube bulk oil tank (tank #2) and the compressor cylinder lube injection system.

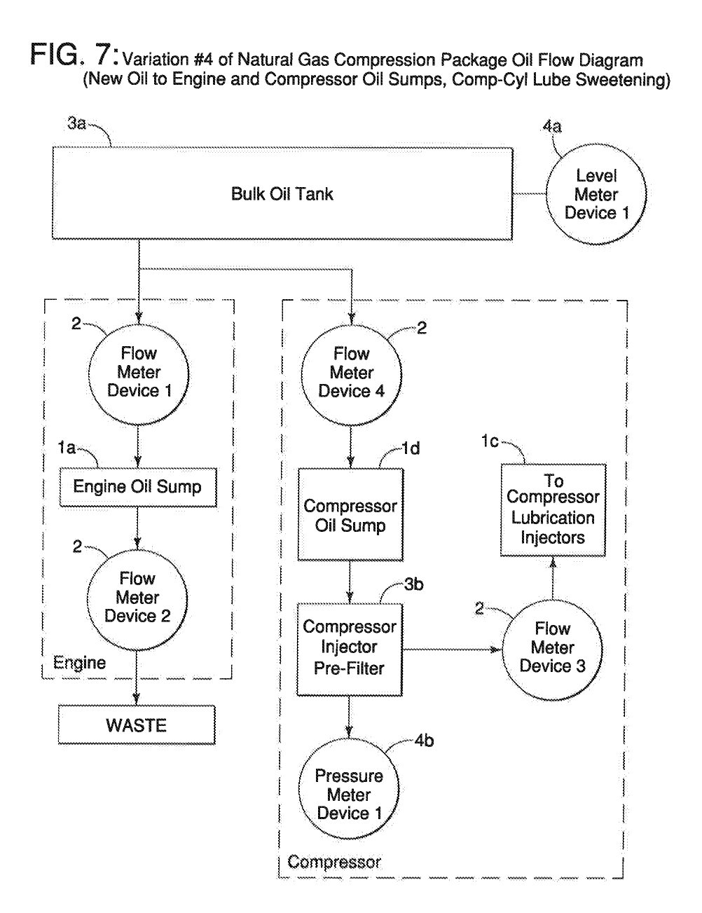

Referring to FIG. 7, an Exemplary Natural Gas Compression Package Oil Flow Diagram is shown (In this exemplary Embodiment, New Oil to Engine and Compressor Oil Sumps, Compressor-Cylinder Lube Sweetening).

### Logic Flow Diagram Item Key:

1) Equipment being monitored for oil consumption:
a) Internal combustion engine, crankcase oil sump level, measured or derived value
b) Internal combustion engine oil consumption rate, rate of oil replenishment, measured or derived value
c) Compressor cylinder lubrication oil injection rate, measured or derived value
d) Compressor or other driven device, crankcase oil sump level, rate of oil replenishment, measured or derived value
   2) Novel oil flow measurement device (invention). Example: a positive displacement gear meter with a direct current (DC) pulse output; the output pulses correlating with a known fluid volume per each full gear revolution, or corresponding pulse. A pulse signal sent to local processing unit (5) for data flow rate computations.
   3) Equipment being monitored for oil consumption:
a) Reservoir tank(s) containing lubricant (oil); provides make-up oil for replenishment of oil consumed by machinery functions, and or leaks.
b) Filter for compressor cylinder lube injector system. Removes contaminates from oil before it enters the lube injector pump and compressor divider block lubrication injectors.
   4) Novel fluid level and pressure measurement devices:
a) Fluid level sensor sending unit, i.e. pressure transducer, etc.
b) Differential pressure (DP) sensor sending unit, i.e. pressure transducer, etc.

### Oil Flow Description:

1) New (virgin) oil is supplied directly from the bulk oil tank to the engine oil sump AND the compressor oil sump.
2) Used (dirty) oil that exits the compressor oil sump is supplied to the compressor cylinder lube injection system.
3) Flow meter devices (invention) are located on each of the inlets of a) the engine oil sump, b) the compressor oil sump, and c) the compressor cylinder lube injection system to measure the flow entering each system.
4) The numerical difference between the total volume of oil entering the engine oil sump and the replenishment volume (flow of oil leaving the engine oil sump) is calculated to be the volume of oil consumption within the engine. When calculated over time, the volumetric flow rate can be determined.
5) The numerical difference between the total volume of oil entering the compressor oil sump and the volume of oil entering the compressor cylinder lube injection system is calculated to be the volume of oil consumption within the compressor. When calculated over time, the volumetric flow rate can be determined.
6) The flow meter device (invention) which is located between the exit of the compressor oil sump and the inlet of the compressor cylinder lube injection system measures both the flow rate of oil exiting the compressor oil sump, AND the compressor lube injection system flow rate. This flow rate value would also be equal to the flow rate of the combined divider blocks (left bank plus right bank).
7) A pressure meter device is used to measure the flow restriction provided by the compressor lube injector pre filter, and can be used to detect a filter malfunction or indicate that the filter must be changed. A maximum pressure differential threshold value across the oil filter inlet and outlet lines can be programmed into the microprocessor unit Dangerous Condition configuration to provide the operator with actionable information, e.g. an indication to change the oil filter. This is very critical in this case since the lube injection system is being supplied with used (dirty) compressor oil. The typical practice is to use two (2) independent pressure gages (liquid filled dial gages) mounted on the filter assembly indicting a) the inlet oil pressure, and b) the outlet oil pressure. This configuration required the gages be routinely monitored, manually reading the gage values, and determining when there is a sufficient pressure differential which would indicate there is a critical flow restriction (e.g. clogged filter media) requiring a filter element replacement.
8) A level meter device is installed on the bulk oil tank to measure:
a) the oil level in the bulk oil tank; and
b) the flow rate of oil out of the bulk oil tank.
   9) Leaks in the system can be detected if the flow rate of oil being drained from the bulk oil tank is measured to exceed the sum of the flow rates of oil entering the a) engine oil sump, b) compressor oil sump, and c) compressor cylinder lube injection system.

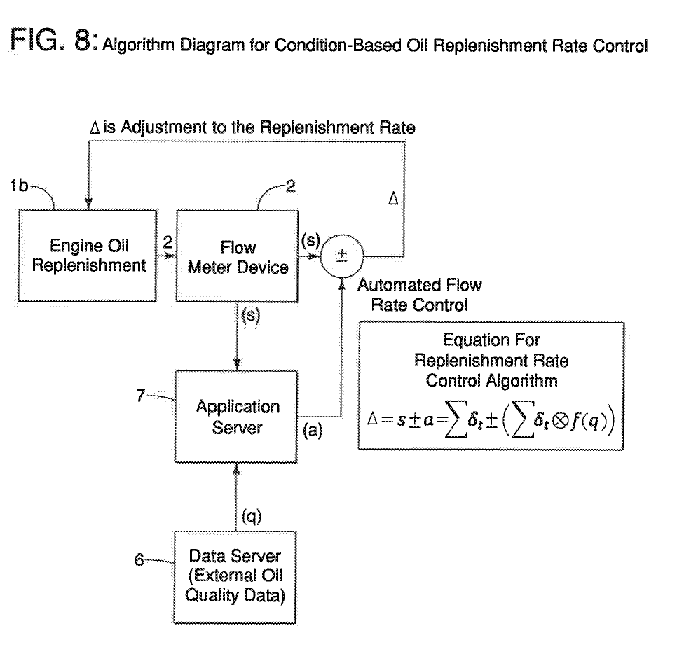

Referring to FIG. 8, an Exemplary Algorithm Diagram for Condition-Based Oil Replenishment Rate Control is Shown.

1. Novel flow rate measurement devices constantly measure the volumetric flow rate of oil into a machine such as an engine or natural gas compressor.
2. This flow rate is transmitted to the Application server 7 where an oil consumption per unit time (e.g. gallons/day) is calculated. This value represents the amount of oil that has entered or exited the machinery over a given time, which is often a key health metric. In the case of natural gas compressors, the compressor cylinders are lubricated at a very precise prescribed rate. Not only is this lubrication rate very difficult to maintain but deviations up or down will cause detrimental effects to the efficiency and health of the compressor.
3. Intelligent controls can be employed in order to create a self-adjusting system, detailed in FIG. 8.

1b: The lube oil is supplied to the mechanical system. In this case the oil source is an engine's crank case (oil sump).

Fluid Flow 2: Oil passes through the flow measurement device, where a volumetric flow rate is reported to the application server 7 via an internet connection.

Ref. no. 7: The application server calculates a flow rate over a period of time and compares it to the prescribed flow rate for that piece of equipment. It then utilizes a control algorithm to adjust a flow control valve on site in order to modify the lube oil flow rate. The algorithms within the application server can also make use of additional oil quality data to better adjust the lubrication rate to optimize efficiency and equipment life.

Ref no. 6: External oil quality data, as provided by other online sensors or an oil analysis laboratory is used to modify the control scheme to adjust for oil cleanliness, chemical properties, contaminates, etc. This results in a smart, learning flow control system that is able to modify the prescribed flow rate based on oil properties.

Feedback (s) is used by the application server to make continuous adjustments (a) to the flow control valve to ensure that the oil lube rate is always optimized. Oil quality data (q) can be used to modify the control algorithm based on oil condition.

The feedback system (s) is also able to alert on malfunction in the case that the desired flow is impossible due to a mechanical failure. This ensures that operators can become immediately aware of an issue before it causes further damage to a given piece of machinery.

According to Another Embodiment of the Invention, an Algorithm for the Averaging and Machine Time-Normalization of Real Time Novel Flow Measurement Device Flow Rate Data is Now Described (On-Engine Installation).

1) Two novel flow rate measurement devices are installed at different points of an engine's oil system.
a. The first novel flow measurement device is installed at the inlet of the engine oil sump; this allows for the measurement of new oil entering the sump (denoted mathematically as m)
b. The second novel flow measurement device is installed at the engine's oil replenishment system outlet, a small amount of used oil is constantly removed from the sump so that it can be replaced with new oil. This replenishment flow rate (mathematically denoted as s) is measured by the second novel flow measurement device.
c. The novel flow measurement devices have the capability of recording machine shutdown/startup data (i.e. if the machine is on or off and other pertinent information).
   2) The raw measured flow rates (m and s) are accumulated over a calendar time (starting at time to and ending at time t₁) window along with corresponding machine shutdown/startup data. This process results in (m_{w} and s_{w}). These totalized oil flow volumes are the summation of each flow data point over the desired time between t₀ and t₁.

$${m}_{w} = {\sum }_{{t}_{0}}^{{t}_{1}} m ; {s}_{w} = {\sum }_{{t}_{0}}^{{t}_{1}} s$$

3) The oil consumption of the engine (c_{w}), which is the result of normal engine operation or an indicator of possible leaks/malfunctions is calculated as the difference between m_{w} and s_{w}.

c_{w}=m_{w}−s_{w}

4) The time interval t₁-t₀ does not take into account the periods of time that the engine is inactive (off), averaging oil flow data over this time period would result in incorrect (artificially low) average oil flow/consumption data if the engine was inactive between t₀ and t₁. In order to remove this calculation error, the time interval is corrected by removing all periods of engine inactivity (denoted mathematically as t_{OFF}). The effective time interval can be calculated as:

t_{eff}=t₁−t₀−t_{OFF}

5) The average effective flowrates for the oil entering the engine's sump (M′), the oil being removed by the replenishment system (S′) and the oil being consumed by the engine (C′) over the time period from t₀ to t₁ can be calculated using the following equations:

$${M}^{′} = \frac{{t}_{1} - {t}_{0}}{{t}_{eff}} \times {m}_{w} ; {S}^{′} = \frac{{t}_{1} - {t}_{0}}{{t}_{eff}} \times {s}_{w} ; {C}^{′} = \frac{{t}_{1} - {t}_{0}}{{t}_{eff}} \times {c}_{w}$$

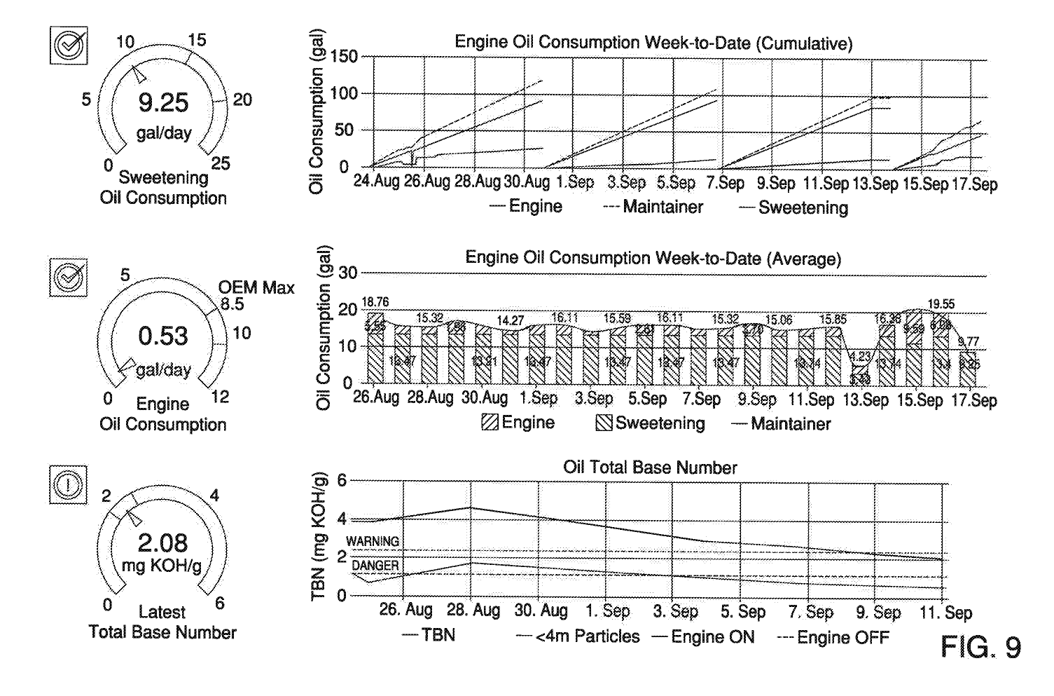

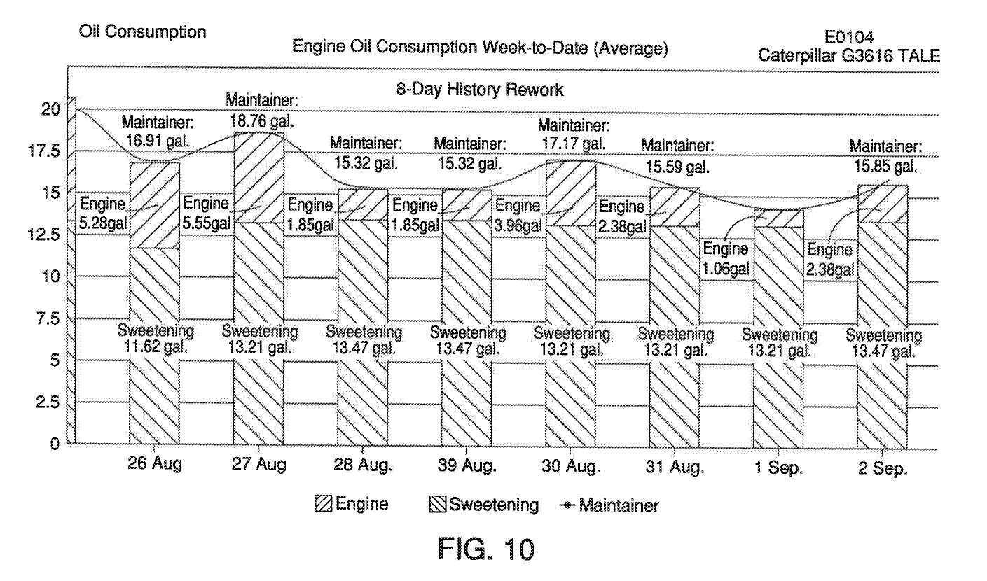

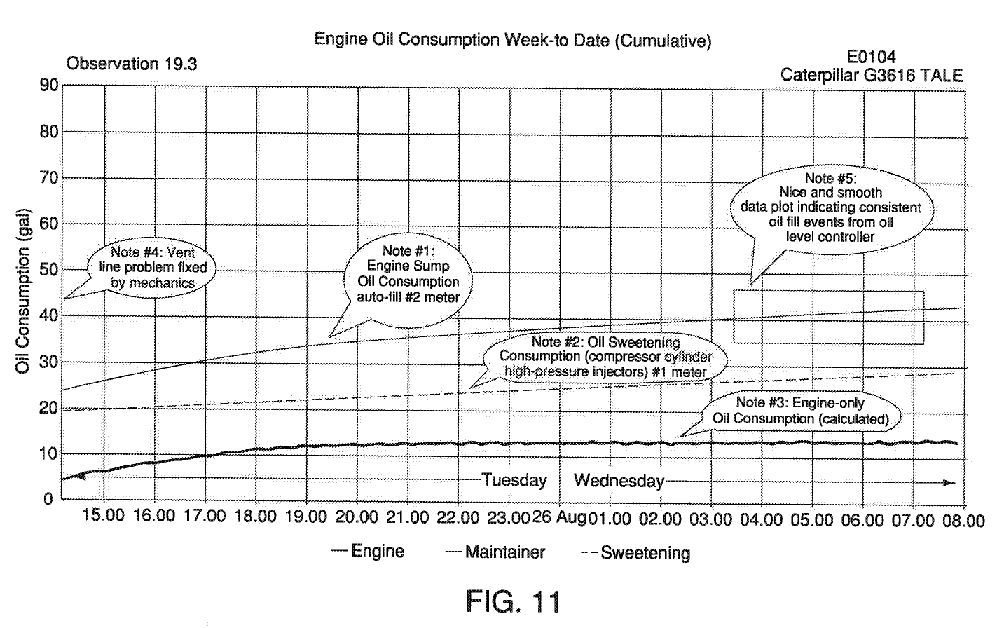

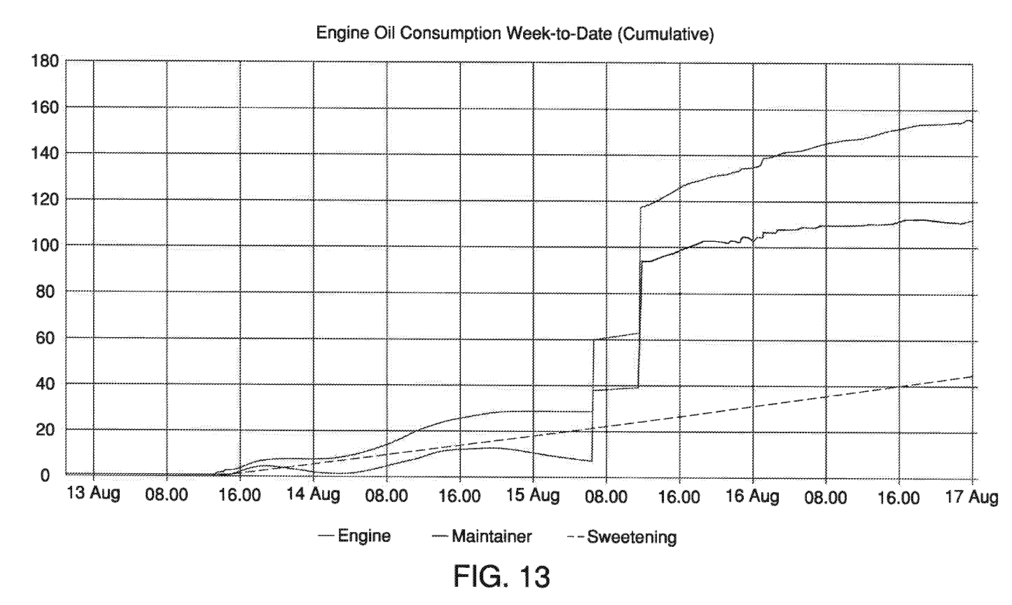

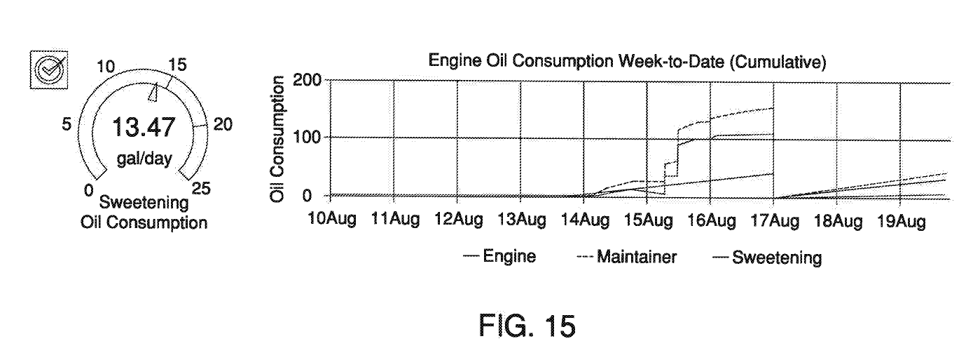

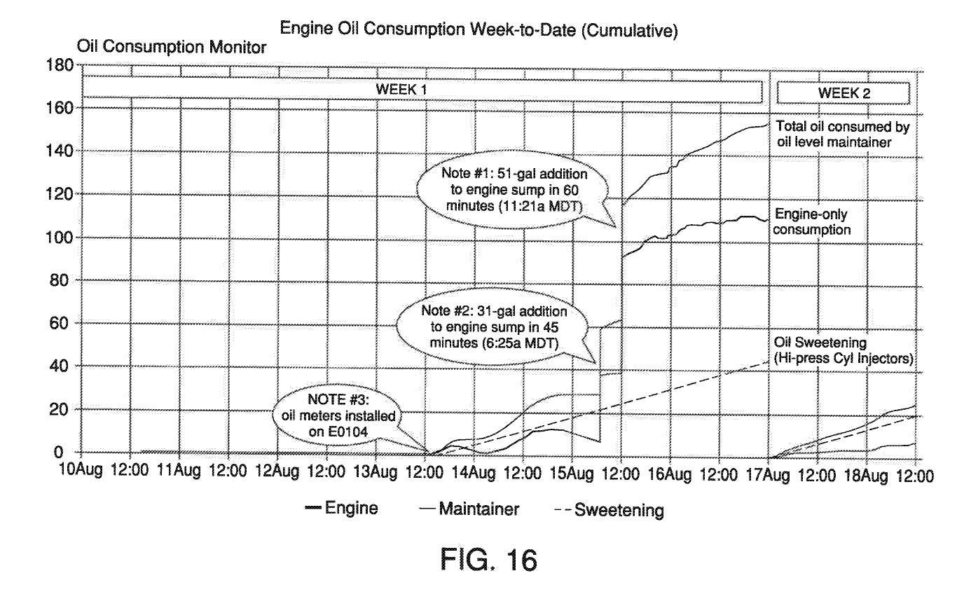

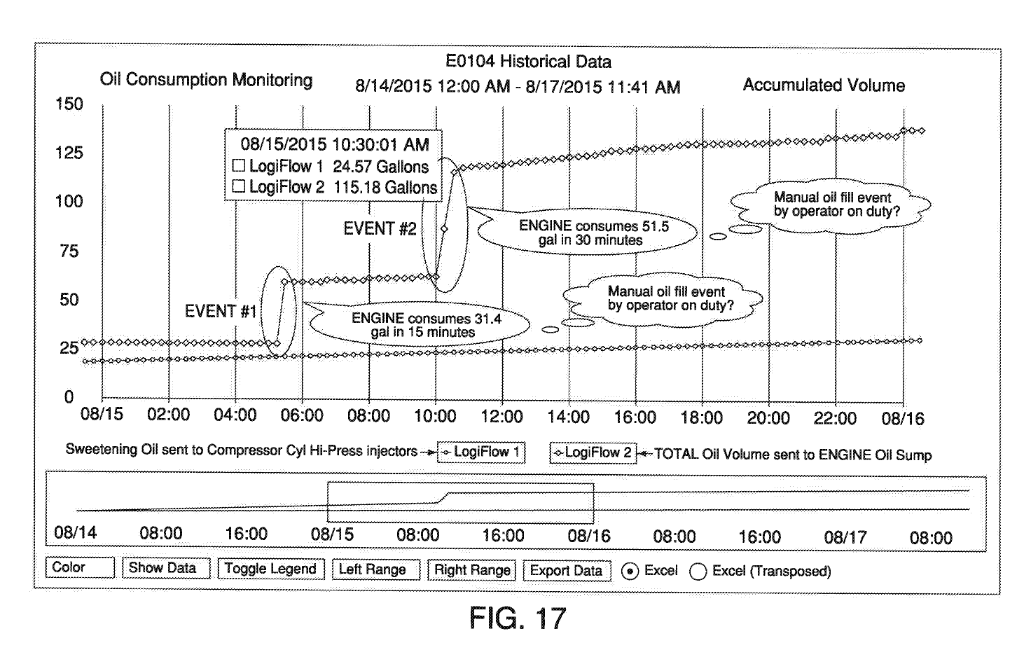

5) Example: Replenishment flow is measured in real time for an entire day (24 hrs), the total flow at the end of the day is measured to be 24 gal. The engine is measured to be ON for 18 hrs and OFF for 6 hrs. Without calculating the effective time period of engine operation (no time correction), the replenishment rate would be reported as 24 gal/day. This is incorrect because there is no oil flow while the engine is inactive (OFF), if a replenishment rate of 24 gal/day is reported, it would lead a customer (i.e. equipment operator/mechanic) to believe that the flow settings are incorrect. By applying the time correction per the calculated effective time, the replenishment rate would be reported as 32 gal/day. This number correctly reflects the engine's replenishment rate settings by adjusting for the engine's OFF time. This enables customers to make real day-to-day comparisons of the engine's operation regardless of the amount of time the engine was OFF.
   Referring to FIGS. 9-23, Exemplary Graphical and Schematic Representations of Various Results of the Exemplary Fluid Flow Monitoring and Management Methods According to Various Embodiments of the Invention Using the Various Exemplary Fluid Flow Monitoring and Management Devices and Systems According to Various Embodiments of the Invention are Shown.

The following description is directed generally to those figures (9-23).

### Oil Consumption Monitoring System (Invention)

### Summary

As installed, the engine mounted oil sump level controller is unreliable, resulting in oil fill events that range in volume from 0.3 gallons to 51 gallons (per event).

Controller unit will unexpectedly trip a ‘low-level’ alarm, thus shutting down the compressor package. RISK—Engine/compressor damage.

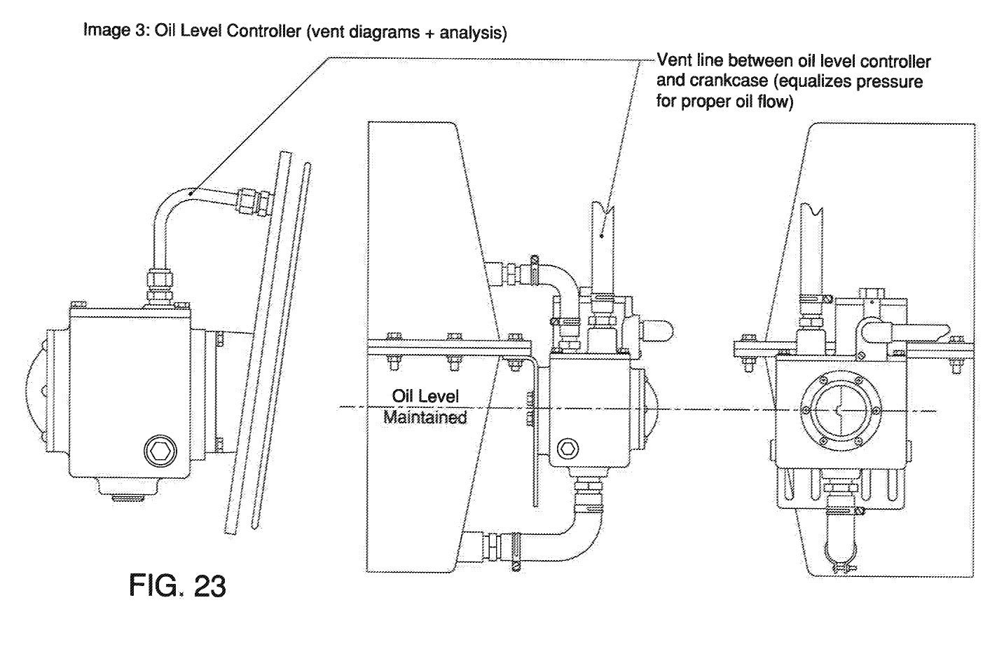

Controller unit is vented to atmosphere vs. requirement to be vented to the engine crankcase, thus creating a slight pressure differential resulting in a burp within the controller valve body, causing a sudden in-rush of oil to the crankcase. [Ref. Image #3] (FIG. 23).

Unreliability of the controller has caused the operators to resort to frequently check the engine oil level in the sump manually via visual inspection of the oil sump dip stick. RISK—Non-timely measurements.

Operators are topping off the engine oil sump manually via opening the 1¼-in controller by-pass valve (gravity feed from oil day tank). RISK—Over filling engine sump causing engine-damaging foamed oil.

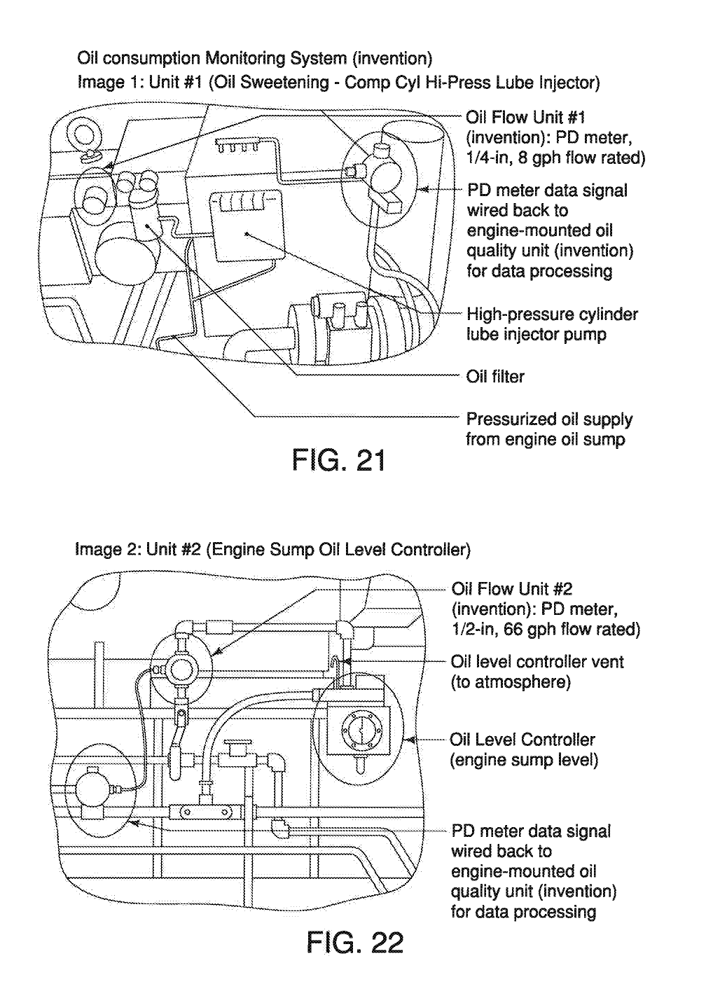

While capturing accurate flow measurements, invention unit #1 (FIG. 21) (engine sump consumption) does not reflect true engine consumption due to the fact that the engine-mounted oil level controller is being augmented by frequent manual oil filling from operators.

Novel invention oil consumption meters (qty 2) are accurately measuring the oil consumption in real-time (cumulative volume measurement reported every 30 seconds).

Oil sweetening system (as monitored by invention unit #2 (FIG. 22) as mounted on compressor) consistently consumes approximately 13.47 gal per day, delivering pressurized engine oil from the engine sump to the compressor cylinder high pressure lube injection system.

Novel invention meter data is processed by the engine mounted microprocessor (novel invention unit microprocessor) and computed results are published to a secure web-based dashboard (novel invention dashboard interface).

### Equipment Details:

Equipment Type: Compressor Driver (Engine)

Equipment ID: E0104

Equipment Model: G3616TALE (16-cyl, turbo assisted lean-burn engine), 350-gal oil sump

Oil Sampling and Quality Monitoring Unit (Invention): Real-time Oil Condition Monitor+Auto Sample Collection (ID: A0009)

Oil Quality Sensors: Oil Temp, Oil Pressure, Dielectric Constant, Oil Density, Oil Viscosity

Oil Flow Meter Unit #1 (Invention) (FIG. 21): Real-time Oil Consumption Meter (Eng sweetening), compressor mounted

Oil Flow Meter Unit #2 (Invention) (FIG. 22): Real-time Oil Consumption Meter (Eng sump-TOTAL), engine mounted

### Background:

### Oil Flow Meter Unit #1 (Invention) (FIG. 21) Oil Sweetening Consumption

Oil flow meter Unit #1 (FIG. 21) (positive displacement (PD) oval gear meter, magnetic pick-up, ¼-in. dia. line) measures the amount of oil delivered to the compressor high-pressure cylinder lube injectors (from the engine sump, pressurized by engine oil pump @˜60 psi).

At unrestricted full flow rate, Unit #1 (FIG. 21) was measured to support a flow rate of 5 gal/hour when supplied by the gravity fed day tank reservoir (reference flow rate only).

Unit #1 (FIG. 21) is physically mounted onto the compressor, located in-line between the final filter and high-pressure injector pump (at the end of

the RECIP compressor frame). [Ref. Image #1] (FIG. 21)

The magnetic pick-up pulse signals generated by the PD meter are sent back to the engine-mounted unit microprocessor via twisted pair shielded signal wire in a Class1 Div 2 rated rigid conduit line.

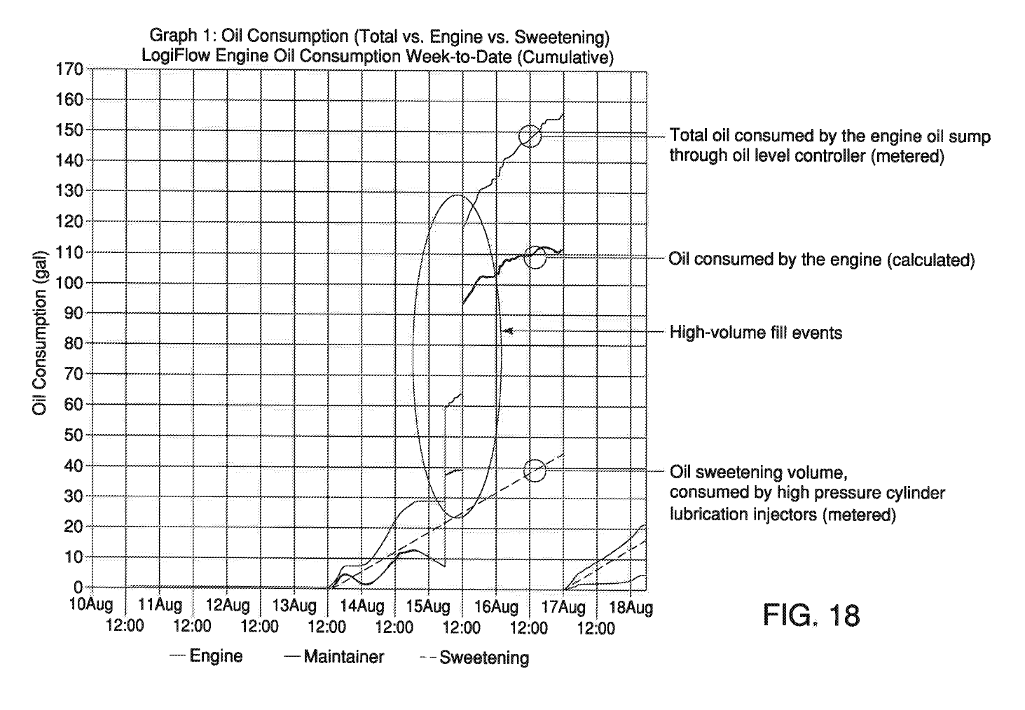

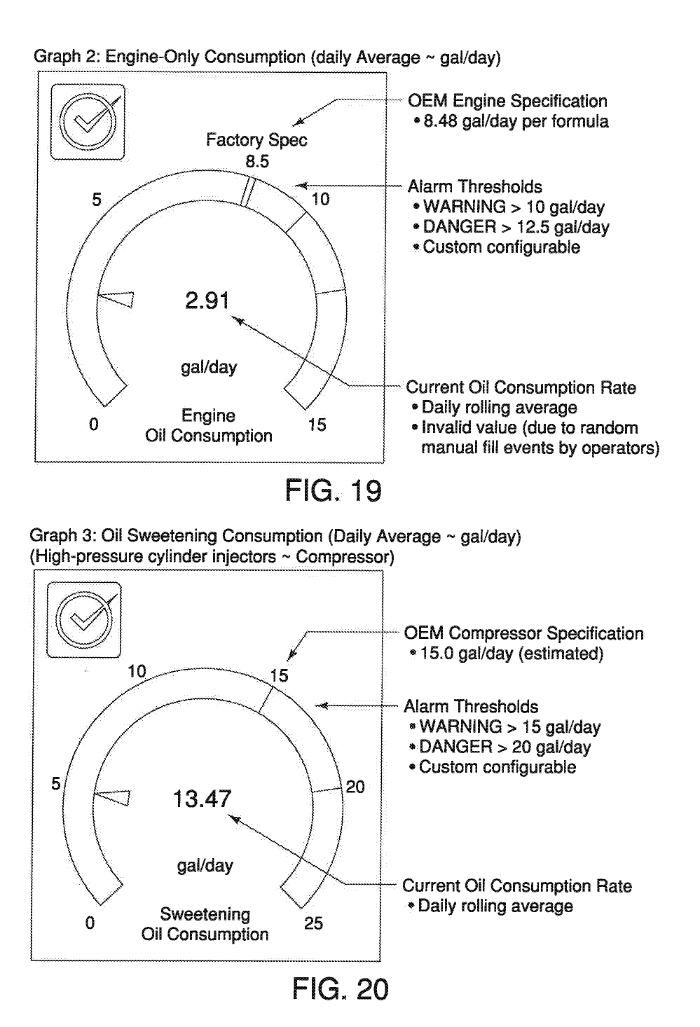

The measured data is processed via custom data signal processing (DSP) algorithms and displayed on company's private and secure web-based dashboard. [Ref. Graph #1 & #2] (FIG. 18 and FIG. 19).

### Oil Flow Meter Unit #2 (FIG. 22). (Invention) Engine Sump Fill Consumption:

Oil flow meter Unit #2 (FIG. 22) (positive displacement (PD) oval gear meter, magnetic pick-up, ½-in. dia. line) measures the amount of fresh makeup oil that tops off the engine sump and is gravity fed by the day tank reservoir (800-gal wall mounted, green bullet tank).

At unrestricted full flow rate, Unit #2 (FIG. 22) was measured to support an oil flow rate of 66 gal/hour to the engine mounted oil sump level controller.

Unit #2 (FIG. 22) is physically mounted onto the engine (CAT G3616), located in-line and immediately upstream of the oil level controller, plumbed into an existing section of rigid ½-in oil line. [Ref. Image #2] (FIG. 22).

The magnetic pick-up pulse signals generated by the PD meter are sent back to the engine-mounted unit microprocessor via twisted pair shield signal wire in a Class 1 Div 2 rated rigid conduit line.

The measured date is processed via custom data signal processing (DSP) algorithms and displayed on company's private and secure web-based dashboard. [Ref. Graph #1] (FIG. 18).

### Observations:

Attribute: Oil Consumption (engine oil)

### Oil Consumption Monitoring System (Invention)

### Observations:

Based on the oil consumption data measured from Unit #2 (FIG. 22) (engine sump), the oil volume per incremental fill event varies widely and erratically, from 0.3 gallons to 51 gallons (per event).

During a 5 hour period on a single day, it was noted there were two (2) large volume engine sump fill events recorded by Unit #2 (FIG. 22) (31-gal at 5:15A, 51-gal at 10:21A).

For reference, the aforementioned unexplained two (2) fill events total 82 gal which is 23% of the 350-gal volume of the engine oil sump).

The oil sweetening rate (measured by Unit #1) (FIG. 21) appears to be consistent with a steady state flow rate at approx. 13.47 gal/day. [This is consistent with an understanding of how the auto-lube system functions and is in direct proportion to the RPM of the compressor crank.]

The erratic behavior of the oil level controller, as observed by the company's invention, has led the O&G plant operators to not trust the auto-sump level control system. Hence, the operators are manually checking the engine sump oil level via visual inspection of the oil sump dipstick several times per shift. When it is apparent that the sump needs to be topped off with new oil, the operators are using the bypass ball valve to add gross amounts of oil to the sump. This requires several visual inspections of the dipstick to insure the sump is adequately topped off.

The engine-mounted oil level controller unit is vented to the atmosphere vs. mfg. requirement of being vented to the engine oil sump. This most likely is the cause for the erratic behavior of the controller unit. Left unattended, this vent plumbing error will likely continue to generate unscheduled engine shut down events. [Ref. Image #3] (FIG. 23).

### Supporting Data:

30-second interval oil consumption data (gal per fill event). [Available upon request]

### Supporting Graph:

Graph 1 (FIG. 18)—Cumulative Oil Consumption (week-to-date, Unit #1, Unit #2 (FIG. 21 and FIG. 22), data plot with WARNING/DANGER thresholds)

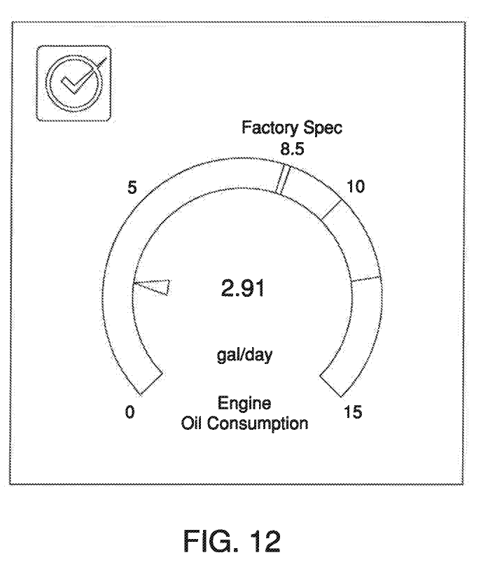

Graph 2 (FIGS. 12 and 19)—Engine-only Sump Consumption (daily average rate, gauge with WARNING/DANGER thresholds)

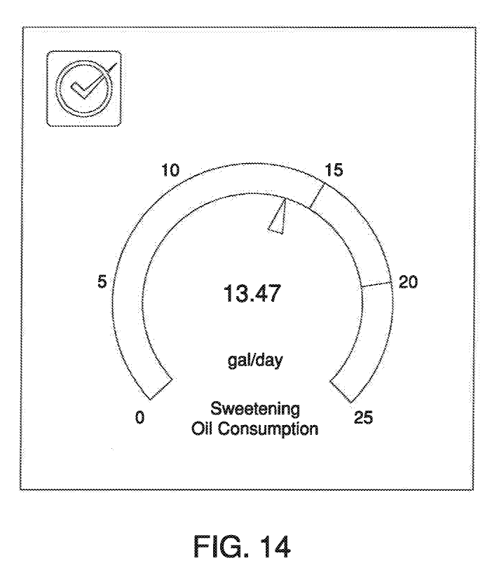

Graph 3 (FIGS. 14 and 20)—Engine Oil Sweetening Consumption (daily average rate, gauge with WARNING/DANGER thresholds)

### Action Taken:

Over the course of previous 3 weeks, company staff embarked on multiple visits to O&G plant site to confer with operators and mechanics on duty to review the oil line routing, oil level controller logic.

The oil line flow rates with oil flow invention positive displacement (PD) meters installed were optimized to minimize any flow restrictions.

The oil flow meter Unit #1 & Unit #2 (FIG. 21 and FIG. 22) (invention) mounting to compressor and engine locations were tweaked.

Class 1, Div 2 data signal wiring runs to the existing oil quality and microprocessing invention unit were run in newly installed rigid conduit on the compressor package frame.

Oil level controller vent plumbing issue was inspected and confirmed.

Company data analysts developed appropriate data algorithms and related web-based dashboard to display actionable information, i.e. real-time consumption rates, historical trends, etc.

### Oil Consumption Monitoring System (Invention)

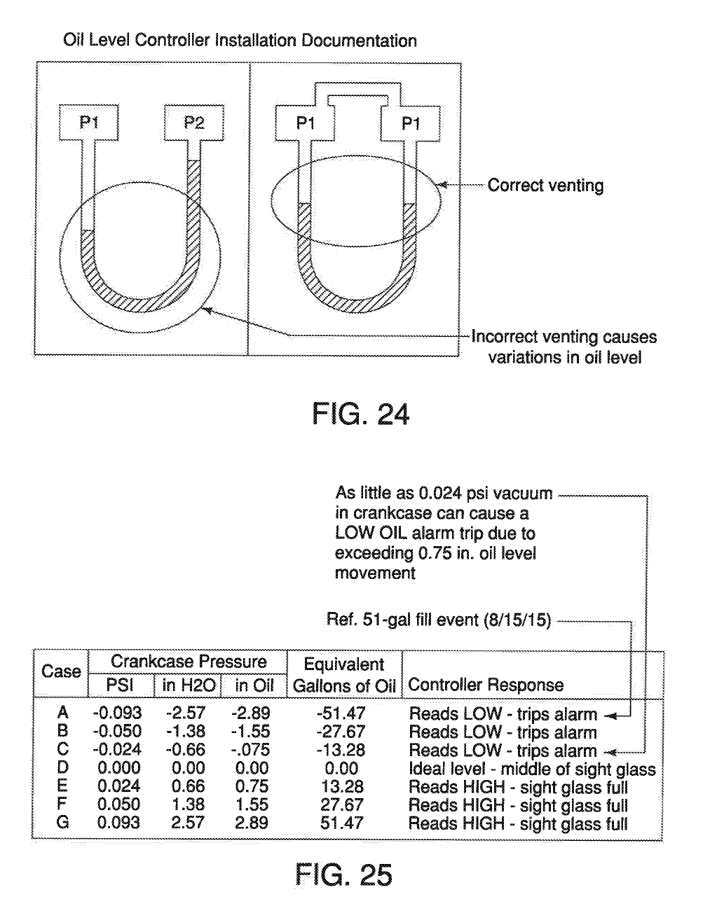

Referring to FIGS. 24 and 25, another factor in the proper operation of an oil controller is equalization. This refers to the pressure inside the oil controller housing matching the pressure inside the crankcase. If the pressures are not equal, the oil level in the controller will not be the same as the oil level in the crankcase. If the pressure in the crankcase is higher than the pressure inside the controller, the oil level in the controller will be higher than the oil level in the crankcase. The controller oil level may look fine, but the crankcase will be low on oil. To counteract this problem, an equalization line is connected between the controller and the crankcase. If this line is too small in diameter or too long in length, it will equalize the pressure between the crankcase and the oil controller.

### Summary of Findings:

Still referring to FIGS. 24 and 25, engine mounted oil level controller installation was reworked by O&G plant mechanics, restoring the unit to proper working order following a faulty initial installation related to venting.

Compressor driver E0104 has not experienced an unscheduled or safety related engine shutdown event since the oil level controller unit was reworked.

E0104 oil sump no longer needs to be manually filled by operator to compensate for failure of oil level controller to reliably maintain the sump oil level.

E0104 oil sump replenishment oil consumption rate has exhibited a consistent and even fill rate over the 7 days since the oil level controller was fixed.

Continued hot weather (>90° F.) induced cyclical load-shedding has not caused any of the repeated engine shutdown events.

Oil quality and flow meter units (invention) have accurately measured, analyzed and monitored the E0104 engine operation and related oil consumption rates resulting in identification (and subsequent fix) of a persistent mechanical engine failure netting a significantly improved engine uptime.

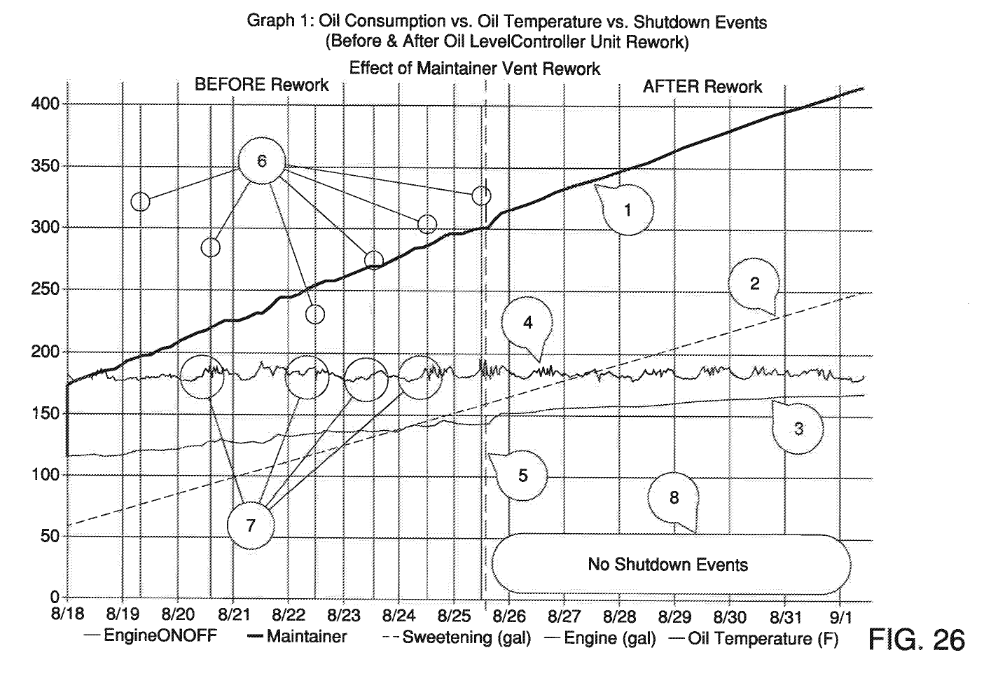

Referring to FIG. 26, graph 1 illustrates oil consumption vs. oil temperature vs. shutdown events (before and after oil level controller unit rework). Reference number 1 indicates engine sump replenishment oil consumption as metered in real-time by Unit #2, with cumulative data in gallons. After oil level controller vent rework (8/25), oil consumption rate is very consistent as indicated by straight plot line.

Reference number 2 indicates engine sweetening oil consumption as metered in real-time by Unit #1, with cumulative data in gallons, and mounted immediately upstream of the compressor high-pressure injector pump. After oil level controller vent rework (8/25), oil consumption rate is very consistent as indicated by straight plot line.

Reference number 3 indicates engine-only oil consumption as computed in real-time by Oil Quality and Consumption microprocessor (invention), cumulative data in gallons (Unit #2 minus Unit #1). After oil level controller vent rework (8/25), oil consumption rate is very consistent as indicated by straight plot line.

Reference number 4 indicates engine oil temperature as measured in real-time by the Oil Quality and Consumption Monitoring Unit (invention). Daily rise in temperature coinciding with ambient outside air temperature. Oscillating temperature corresponding to the load-shedding modulation deployed to maintain engine temperature.

Reference number 5 indicates oil level controller unit rework as repaired by O&G plant mechanics (fix vent, reposition controller body).

Reference number 6 indicates engine shutdown events as monitored by Oil Quality and Consumption Monitoring Unit (invention) Dangerous Condition algorithm generated flag. Corresponds to first load-shedding cycle on hot days.

Reference number 7 indicates LOW OIL LEVEL alarm trigger. Oil level controller oil level switch shuts down engine setting off Oil Quality and Consumption Monitoring Unit (invention) ENGINE OFF alarm notification, event logged by Oil Quality and Consumption Monitoring Unit (Invention). Engine shuts down on FIRST load-shedding cycle. After operator floods sump to re-start, oil level controller LOW LEVEL switch is not tripped on subsequent load-shed cycles due to overfilled sump.

Reference number 8 indicates Post Oil Level Controller Unit Rework. No engine shut down events experienced. All oil consumption rates are measured as being very consistent. Continued hot weather (<90° F.) induced load-shedding events have not caused any previously witnessed engine shutdown events.

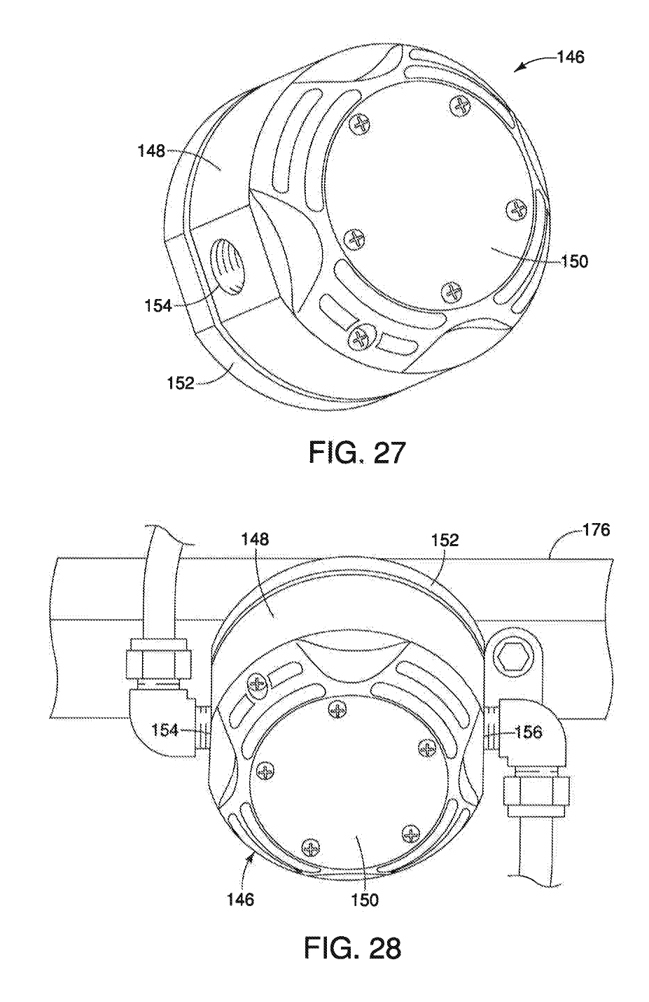

Referring to FIG. 27, a fluid flow monitoring and management device (or fluid flow measurement device or flow measurement device or flowmeter) 146 is illustrated according to an embodiment of the invention. An exemplary fluid flow measurement device 146 includes a fluid flow body 148 and a sensor (or sensor body or sensor device or flow sensor) 150 secured to the fluid flow body 148. The fluid flow body 148 includes a base 152 and an opening 154 to receive a fluid flow (opening 154 may be an inlet or outlet depending on the direction of the fluid flow). Sensor 150 is secured to a top portion of the fluid flow body 148.

Referring to FIG. 28, fluid flow measurement device 146 is illustrated secured to a structure 176, an exemplary structure being a compressor package. The fluid flow measurement device 146 is illustrated mounted in-line with a fluid flow through a fluid line (not referenced). The fluid line is secured into opening 154 of the fluid flow body 148 and into opening 156 (referenced for the first time). It should be understood that this inventive fluid flow measurement device 146 is capable of measuring fluid flow in any fluid flow direction, that is, fluid flow measurement is non-directional with metering accuracy not being affected by fluid flow direction.

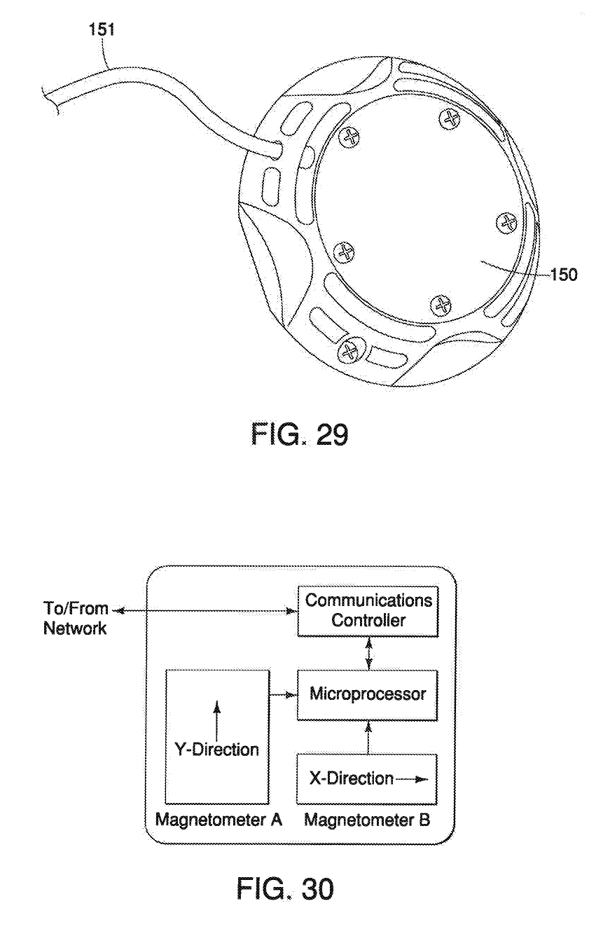

Referring to FIG. 29, sensor (or sensor device) 150 is illustrated, without the fluid flow body 148, and includes the electronic measurement equipment described more thoroughly below. Sensor 150 includes a sensor line 151 for transmitting signals and data to and from a network.

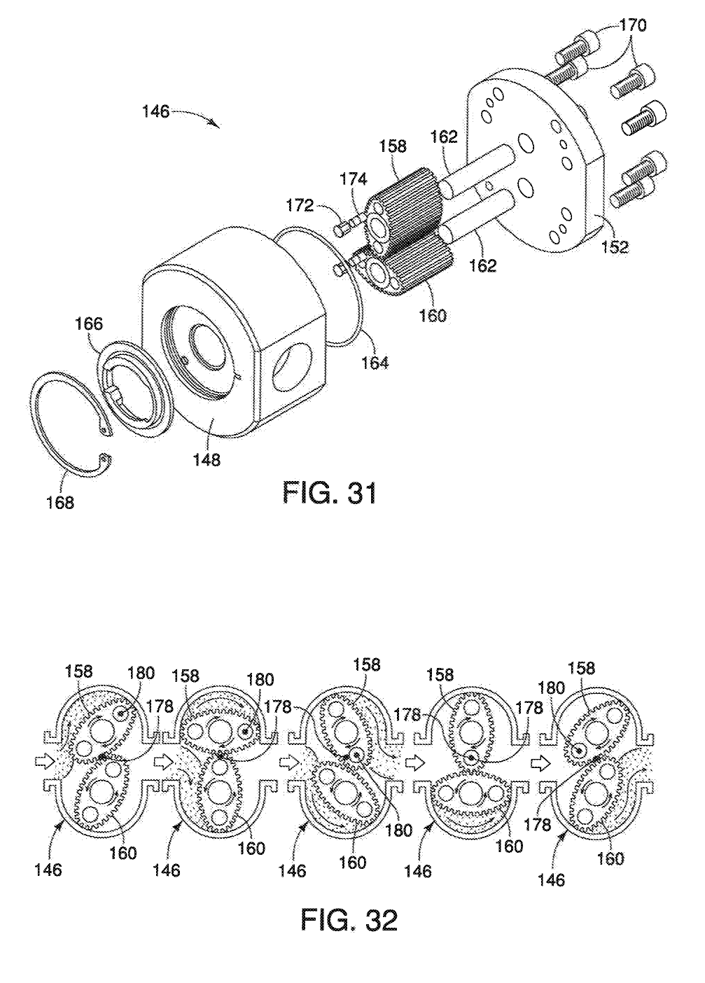

Referring to FIG. 31 (before describing FIG. 30), an exploded view of the fluid flow body 148 of the fluid flow measurement device 146 is illustrated. A circlip 168 and cam 166 are provided on one side of the body 148 and o-ring 164 is provided on an opposite side of the body 148. Two oval rotors (gear rotors) 158 and 160 are in a meshing relationship over rotor shafts 162. Rotor shafts 162 are secured in base 152. A magnet housing 172 is provided over a magnet 174 in one end of one rotor 158. The two oval rotors (gear rotors) 158 and 160 are housed in body 148. Screws 170 secure all the structures together.

It should be understood that the gear rotor function within the fluid flow monitoring and management device is capable of handling a wide range of viscosities, for example, water (1.0 centipoise @ 20° C.) to peanut butter (250,000 centipoise at 20° C.).

Referring to FIG. 30, a sensor 150 layout is illustrated. The sensor 150 features two magnetometers which are oriented 90 degrees apart. A vertically oriented (or along y-axis or direction) magnetometer is referenced as magnetometer A and a horizontally oriented (or along x-axis or direction) magnetometer is referenced as magnetometer B. An exemplary magnetometer is a MEMS-based magnetic field sensor. The sensor 150 also features a processor (microprocessor) that reads outputs from the two magnetometers and processes them into a defined angular position of the flow meter gear rotor (or gear rotor) 158. As the gear rotor 158 spins (rotates) under the influence of fluid flow, the processor tracks the position of the gear rotor 158 over time and is able to produce an angular velocity (rotation speed) of the gear rotor 158 as well as count the degrees traveled since the last data transmission. Real-time volumetric flow rates can be calculated extremely quickly using the angular velocity of the sensor rotor 158. That is, this fluid flow monitoring and management device 146 enables the positive displacement of oval gear rotors 158 and 160 to provide the capability to read flow direction of the fluid flow which is not currently possible. The sensor device 150 further includes a communications controller provides communication between the processor (microprocessor) and a network 151. An exemplary network 151 can include a data bus network (Modbus, CAN Bus, etc.) or to a TCP/IP network for internet connectivity.

Referring to FIG. 32, a method of the mechanical operation for the fluid flow monitoring and management device 146 is illustrated. FIG. 32 illustrates the progression of the fluid at five different stages advancing as one moves from left to right. Fluid that passes through the body 148 causes both rotors 158/160 to turn. The fluid can enter from either side of the body 148. In this embodiment, fluid enters from the left. Rotor 158 (the active rotor) with the magnet 180 (referenced as 174 in FIG. 31) rotates in the clockwise direction. The meshing relationship between rotors 158/160 results in rotor 160 moving in a counterclockwise direction. Rotor 158 (the active rotor) with the magnet 180 provides magnetic signals as the magnet passes the magnetometers in the sensor 150.

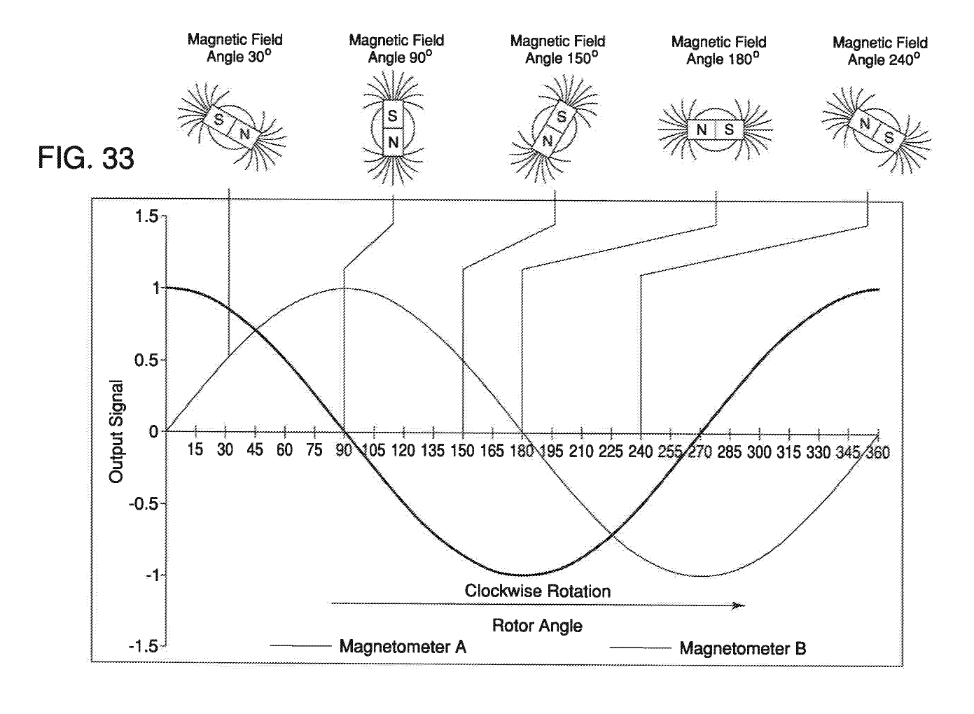

Referring to FIG. 33, the progression of the magnetic field corresponding to the progression of magnet 180, respectively, in FIG. 32 is illustrated. Moreover, FIG. 33 illustrates a graphical progression of the output of sensor 150 corresponding to the progression of magnet 180, respectively, in FIG. 32 (and magnet field of in this FIG.) is illustrated. Accordingly, the orientation of the first magnetic field, and first output, corresponds to the first position of the magnet 180 of FIG. 32, and the orientation of the second magnetic field, and the second output, corresponds to the second position of the magnet 180 of FIG. 32, etc. The respective sine waves represent the respective magnetometer output. The sine wave beginning at the point (0,0) represents the output for magnetometer A (vertically oriented) and the sine wave beginning at the point (0,1) represents the output for magnetometer B (horizontally oriented). The intersection of the sine waves by the vertical lines represent the respective output points of each magnetometer.

Still referring to FIG. 33, every angle or curve of the graph corresponds to a unique output combination of Magnetometer A and Magnetometer B. Forward fluid flow (as shown in FIG. 32) will always produce the same output signal progression (shown above). The sensor 150 of the fluid flow monitoring and management device 146 can determine the exact angular position of the rotor 158 based on the comparison of Magnetometers A and B. The processor contained within the sensor device 150 is programmed to “know” what numbers to expect next based on the curves shown above.

It should be understood that reversed fluid flow will produce a different output signal progression that is detected by the processor of the sensor device 150. The sensor device 150 provides a digital output that includes the total degrees traveled by the rotor since the last data transmission and the average angular velocity (degrees or radians per second) of the rotor 158 since the last transmission. Volumetric flow rates can be calculated both external or internal to the sensor device's 150 on-board processor.

It should be understood that the sensor 150 electronics of the fluid flow monitoring and management device 146 facilitates measurement of the magnetic field as the gear rotors spin. This approach provides a much finer resolution and allows for flow rates to be reported in real-time (second-by-second), as opposed to having to wait a long period of time. Since the rotor speed and direction are measured, any backwards flow that may occur can be cancelled out automatically on-device via firmware residing on the rugged, low power edge processor within the sensor 150.

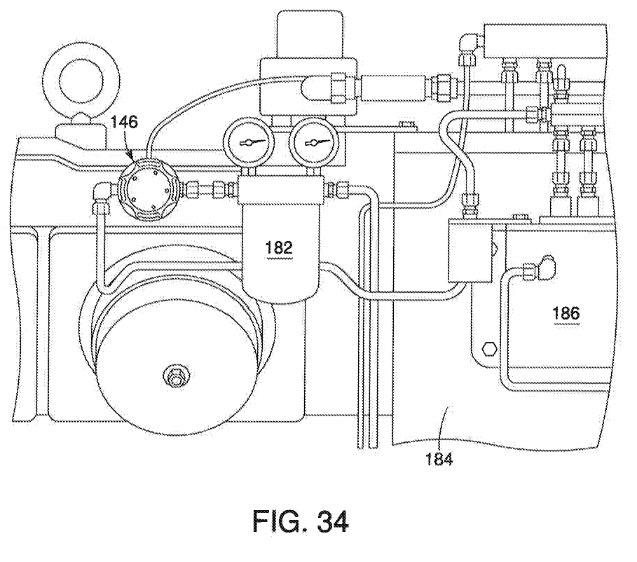

FIG. 34 illustrates an exemplary embodiment of the fluid flow monitoring and management device 146 mounted in a fluid flow (or plumbed) of a compression asset or package which includes lube oil filter 182, compressor 184 and force-feed lube pump 186.

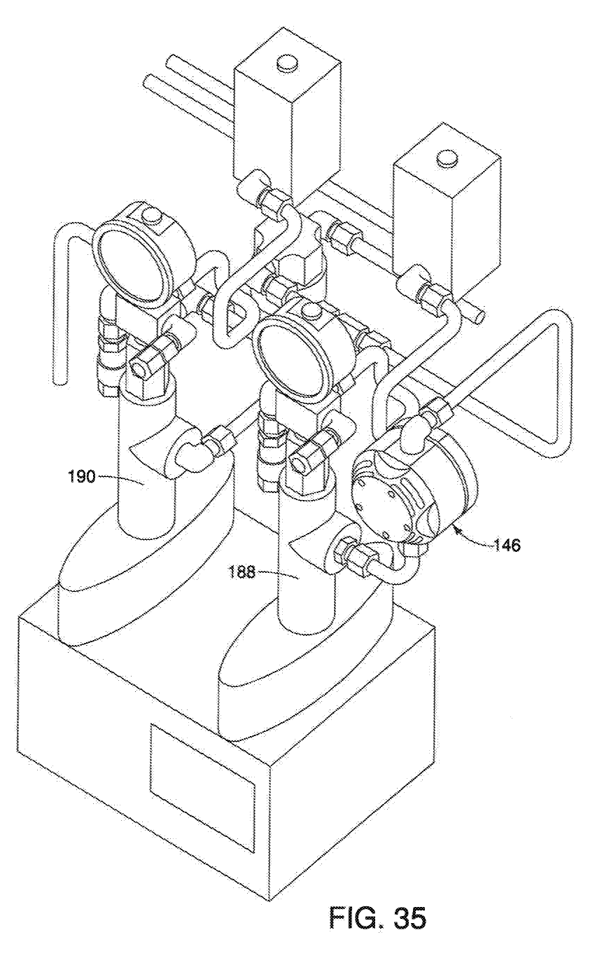

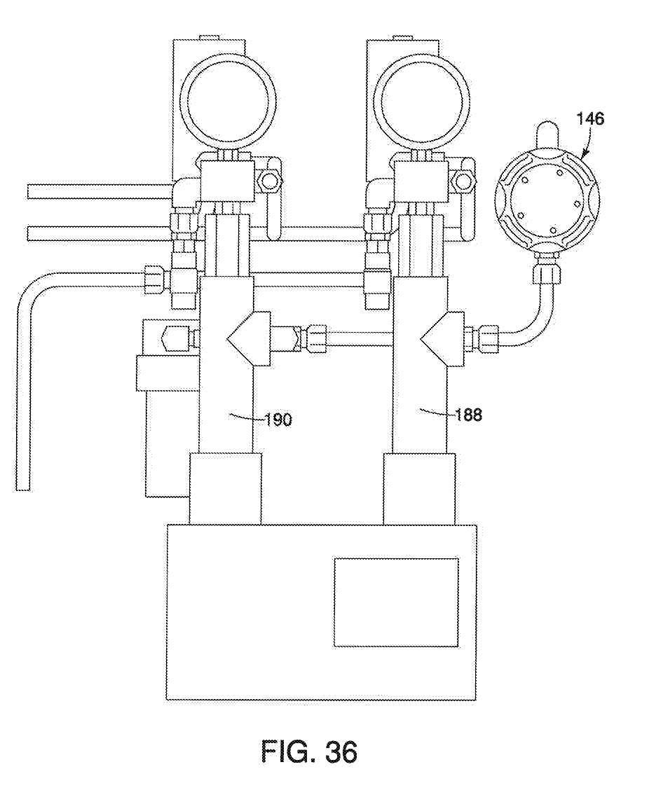

FIGS. 35-36 illustrate an exemplary embodiment of the fluid flow monitoring and management device 146 mounted in a fluid flow (or plumbed) of a force-feed lubrication system which includes feed oil to power cylinders 190 and feed oil to compressor cylinders 188. In this embodiment, device 146 is in fluid flow with the feed oil to compressor cylinders 188. However, the fluid flow monitoring and management device 146 can also be mounted in a fluid flow with the feed oil to power cylinders 190.

It should be understood that prior art fluid flow meter devices or sensors are dependent on a K factor (K-factor) to measure the fluid flow. That is, prior art fluid flow meter devices output a pulse wherein an exemplary K factor=400 pulses per gallon. However, the inventive fluid flow monitoring and management device (or sensor 146) is not dependent on a K factor. This is due to the fact that the novel fluid flow monitoring and management device 146 circuitry is able to read the angular velocity (rotation speed) of the rotor gear in real time. For example, in one embodiment, the novel sensor 146 circuitry is able to read the angular velocity of 2π rad/sec, which in this embodiment, equals 1 pulse/sec. This approach allows the novel sensor 146 to measure and report much smaller flows as their resolution is much smaller, that is, 0.2π rad/sec=0.1 pulse/sec, meaning that the prior art flow meter device would take 10 times as long to produce a pulse.

It also should be understood that the novel fluid flow monitoring and management device 146 is also able to measure reversed fluid flow, as stated previously, when the rotor gears spin backwards. In contrast, using a prior art fluid flow meter design, the pulses between fluid flowing in forward direction and flowing in a backward direction are indistinguishable.

The novel fluid flow monitoring and management device 146 also permits technicians using a handheld device to adjust the force-feed lube rate in real time since the flow rate changes will register almost instantly to the novel sensor 146 as opposed to after several pulses (which could take minutes, or even hours for a prior art device.

The importance of having real time measurements of fluid flow rates simply cannot be overstated. In compressor lube system applications, the appropriate compressor cylinder lubrication rate is essential to ensure the proper compressor operation, over-lubrication and under-lubrication result in reduced compressor efficiency and lower component life. Knowing the amount of lubricant being used on a compressor allows for two things: prevention of shutdown because of under lubrication and wasted money due to over lubrication.

For example, under-lubrication:

1. If a compressor is under lubricated, maintenance costs are going to increase exponentially.

2. To put it very simply, without proper lubrication, parts wear out much more quickly causing the need for replacement.

3. To replace any parts, you will need to completely shut down the compressor, causing work to stop and money to be lost.

4. You will likely have damage to other parts throughout the compressor, and there is the added potential for emissions issues as well.

For example, over-lubrication:

1. When over lubricated, a compressor may continue to function well. But there is still a cost, and it adds up to hundreds of dollars per week.

2. The break in rates for most natural gas compressors is 150 to 180% of the needed level at a normal operating rate.

3. If you have not adjusted that setting, you're using between 1.5 and 1.8 times the oil you need.

4. Added to oil expense, which is the number one operating expense compressor owner/operators list, you add the expenses associated with valve deposits and breakage, premature packing failure and error and downstream contamination and measurement error.

Use of the fluid flow monitoring and management device 146 invention is not limited to the lube oil for an engine or a compressor. It can also be used with gear oil used in a gearbox, e.g. wind turbine gear box, speed reducer gearbox of a cooling tower fan, etc. Moreover, the fluid flow monitoring and management device 146 can also be used for other fluids, for example, hydraulic fluid, transmission fluid, process chemicals, water, food and beverage fluids, slurries, etc.

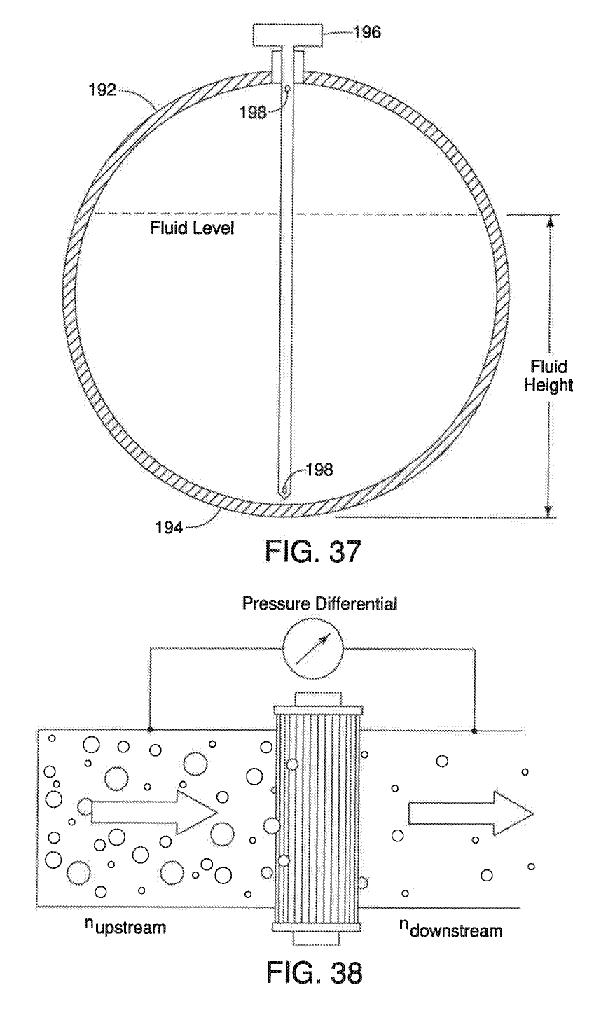

Referring to FIG. 37, an exemplary level measure device (level meter or fluid level meter) is illustrated according to embodiments of the invention. Volume is derived from fluid column height and vessel cross section. For example, the fluid level meter is installed in a fluid tank 192. Fluid pressure measurement is facilitated by the transducer 198 at the bottom of the tank. Atmospheric pressure measurement is facilitated by the vent tube 196 located above the fluid level, this allows the fluid level meter to work in situations where the tank is pressurized. The pressure difference between the fluid pressure measurement and the atmospheric pressure measurement, plus the density of the fluid is used to calculate a very precise fluid height. The fluid height coupled with the tank cross section can be used to calculate the precise volume of the fluid in the tank.

The fluid level meter in this embodiment can communicate with the internet via wired (RS-485) or wireless (900 Mhz RF) data link. Consequently, the precise volume of the fluid in the tank is calculated in real time terms.

Moreover, flow rate can also be calculated by looking at the tank level change over time and the calculation presented in real time.

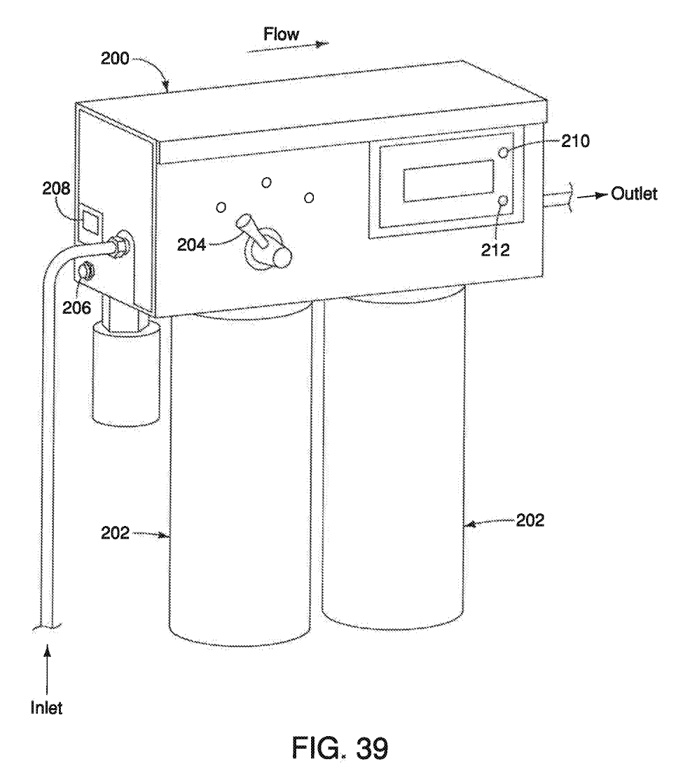

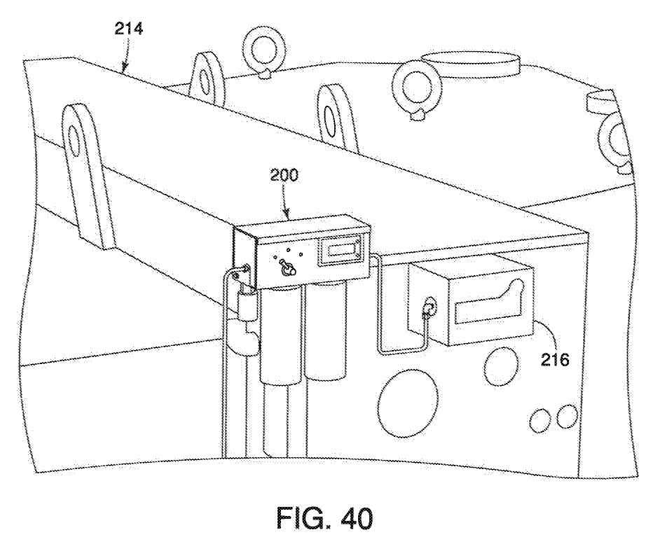

Referring to FIGS. 39-40 generally, and to be discussed more thoroughly subsequently (FIG. 38 describe subsequently), an exemplary fluid flow monitoring and management system that includes a differential pressure measurement device is shown according to one embodiment of the invention and illustrated being applied to a compressor lubricant filtration systems according to one embodiment of the invention.

Assuming that the fluid type does not change, differential pressure measurement across a filter is affected by two main factors, flow rate and filter loading. A brand new filter with a low flow rate should display a very small differential pressure while a brand new filter with a high flow rate may display significantly higher differential pressures.

This differential pressure measurement, when applied to filters, can be used by the inventive data processing system to determine the remaining useful life of an oil filter, in real time, as well as identify situations in which the filter has failed, in real time, which may contribute to a machine failure/downtime event.

Referring to FIG. 38, details for how the pressure across a filter is to be measured is illustrated. Pressure sensors are connected to the fluid flow through the novel fluid flow monitoring and management device 146 described above and fluid flow through the novel filter configuration/design discussed more thoroughly below in order to facilitate real-time online monitoring of filters and filter health.

Again referring to FIGS. 39 and 40, a lube oil conditioning/quality monitoring system (or monitoring device or fluid flow monitoring and management system) 200 for compressor force-feed lubrication systems is more thoroughly described. Referring to FIG. 39 monitoring system 200 packages the flow measurement system (or fluid flow monitoring and management device 146) and the differential pressure measurement device, discussed above, into an online monitoring package which is capable of monitoring the force-feed lube rate (via flowmeter) and the filter life expectancy (flow meter+differential pressure meter). It is also connected to the same data network as discussed above for real-time transmission and can operate in conjunction with the described novel technologies, or as a standalone system.

Still referring to FIG. 39, the fluid flow monitoring and management system 200 includes the following structures: two high efficiency spin-on filters 202, a bypass valve 204, an edge processed data output 210, QR code for chain of custody 208, manual (push button) oil sample port 206 for inlet, and online monitoring 212 which can include filter life and fluid flow rate. It should be understood that cylinder structure (not referenced) extending between inlet and filters is a fluid sample bottle to receive fluid samples for testing. It should also be understood that on opposite side of the management system 200 is another manual (push button) oil sample port for outlet.

Referring to FIG. 40, the management system 200 is shown mounted to an RECIP compressor 214 and is plumbed to a force feed pump 216 which supplies oil to divide blocks of the compressor 214. The management system 200 employs edge processing to calculate filter remaining useful life (RUL) and force-feed lube rate to display to the user via an on-board vacuum fluorescent display (VFD). Such a display is highly tolerant to vibration and heat. Analytics alerts are also available to the end user via email and SMS alerts.

Compressor cylinder force-feed lubrication systems require a steady supply of high quality, highly filtered oil to prevent premature compressor failure and/or reduced operating efficiencies. Most filtration systems are incapable of removing most of the 1-2 micron particles that will cause damage to the high tolerance lubrication system components such as the lube divider blocks and force-feed pump assemblies.

Moreover, filtration system maintenance is often ignored because the compressor has to be shut down prior to changing filters. However, the novel the management system 200 features a bypass valve 204 that allows the pressurized oil flow to be momentarily diverted around the filters, allowing the machine to remain in operation while the filters are changed. These force-feed systems can be supplied by the engine (sweetening) or from a secondary source such as the compressor oil supply.

Depending on the source of the lube oil (engine sweetening or compressor), the oil may be significantly dirtier than it needs to be for the compressor. Filter life tracking and calculation of filter RUL provide operators with the ability to change filters based on their RUL instead of a schedule.

MANUAL oil sampling ports 206 located on the inlet and the outlet (not shown or referenced) of the management system 200 allow for operators to obtain lab analysis of the incoming oil vs. the outgoing oil to ensure that the management system 200 is adequately conditioning the force-feed lube oil.

Since the force feed lube rate is measured in real-time by the the fluid flow monitoring and management device 146 in the management system 200, the management system 200 provides an excellent high accuracy crosscheck to the existing Digital No Flow Timer (DNFT) technology that is already used by the compression industry. DNFT devices mount on the compressor divider block and alarm if a divider block shuttle valve does not actuate at a certain speed. Other technologies, such as Monico Lubewatch, measure the divider block pulses/min and calculate a force-feed lube rate. This approach assumes that no wear has occurred within the lube system which is a poor assumption to make in the event that the lube oil cleanliness is not up to the appropriate standards.

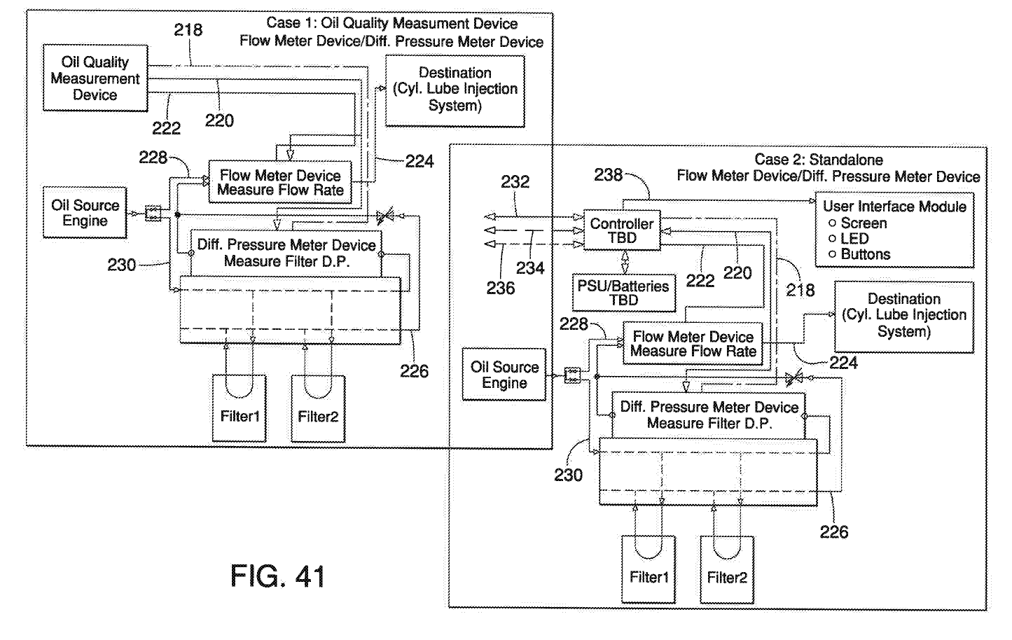

Referring to FIG. 41, a diagram of the layout of the fluid flow monitoring and management system 200 which was described with respect to FIGS. 39-40. Lines 218 represent an analog sensor signal (0-5 VDC). Lines 220 represent excitation voltage to sensors. Lines 222 represent an analog sensor signal (pulse output). Lines 224 represent fluid flow to the injection system. Lines 228 represent filter bypass. Lines 230 represent dirty oil to filters. Lines 226 represent clean oil to filters. Line 232 represents system power (supplied power) (TBD, VDC). Line 234 represents wireless data link (RF 900 MHz/Cell) (serial data link). Line 236 represents wired data serial link (RS 485). Line 238 represents communication between controller and User Interface Module.

In compliance with the statute, embodiments of the invention have been described in language more or less specific as to structural and methodical features. It is to be understood, however, that the entire invention is not limited to the specific features and/or embodiments shown and/or described, since the disclosed embodiments comprise forms of putting the invention into effect.

1. A fluid flow monitoring and management device comprising:
a fluid flow body;
a gear rotor in the fluid flow body; and
a sensor secured to the fluid flow body, the sensor comprising a magnetometer and configured to output angular velocity of the gear rotor.

2. The device of claim 1 wherein the magnetometer comprises a first magnetometer and wherein the sensor comprises a second magnetometer.

3. The device of claim 2 wherein the first and second magnetometers are oriented in a 90-degree relationship.

4. (canceled)

5. The device of claim 1 wherein the at gear rotor is oval shaped and has only a single magnet in one end of the gear rotor.

6-9. (canceled)

10. The device of claim 1 wherein the sensor is configured to output rate of fluid flow in real time.

11. A fluid flow monitoring and management system comprising:
a fluid flow meter;
a filter comprising an inlet and an outlet; and
a differential pressure measurement device coupled to the inlet and outlet of the filter; and
a fluid level controller unit comprising a pressure equalization device.

12. The system of claim 11 wherein the differential pressure measurement device is configured to output the remaining useful life (RUL) of the filter.

13. The system of claim 11 wherein the fluid flow meter is configured to output the rate of fluid flow in real time.

14. The system of claim 11 wherein the fluid flow meter comprises:
a gear rotor comprising a magnet; and
a sensor comprising a magnetometer in a sensing relationship with the magnet.

15. The system of claim 14 wherein the sensing relationship establishes the capability to measure an angular position of the gear rotor.

16. The system of claim 14 wherein the sensing relationship establishes the capability to measure an angular velocity of the gear rotor.

17. The system of claim 11 wherein the fluid flow meter is configured to output the rate of fluid flow in real time.

18. A fluid flow monitoring and management method comprising:
positioning a fluid flow meter in a fluid flow; and
outputting the rate of the fluid flow in real time without relying on a K factor.

19. The method of claim 18 wherein the fluid flow meter comprises:
a gear rotor comprising a magnet; and
a sensor comprising a magnetometer in a sensing relationship with the magnet.

20. The method of claim 19 wherein the sensing relationship establishes the capability to measure an angular velocity of the gear rotor.

21. The method of claim 19 wherein the sensor is configured to output the rate of fluid flow in real time.

22. The method of claim 18 further comprising:
positioning a filter in the fluid flow, the filter comprising an inlet and an outlet; and
determining a differential in pressure between the inlet and the outlet of the filter.

23. The device of claim 1 wherein the sensor comprises a MEM-based magnetic field sensor.

24. The device of claim 1 wherein the sensor is configured to measure the volume of the fluid flow without relying on a K factor.

25. The device of claim 1 wherein the sensor is configured to measure the volume of the fluid flow without relying on a pulse per volume determination.
